(19) United States
(12) Patent Application Publication
(10) Pub. No.: US 2025/0375559 A1
(43) Pub. Date: Dec. 11, 2025

(54) POD PUMP FLUID MANAGEMENT WITH END OF DRAIN AND OCCLUSION SYSTEMS AND METHODS

(71) Applicant: Gambro Lundia AB, Lund (SE)

(72) Inventors: Belur Shanthakumar Shashanka Jain, Bangalore (IN); Vinayaka Harakabhavi Mutt, Bangalore (IN); Anoop Thirumattathil Ashokan, Alappuzha (IN); Sunkad Sachin, Bangalore (IN); Ravikumar Hemanth Hiriyur, Bangalore (IN)

(21) Appl. No.: 19/232,490

(22) Filed: Jun. 9, 2025

(30) Foreign Application Priority Data
Jun. 10, 2024 (IN) 2024/41044759

(51) Int. Cl. A61M 1/28; A61M 1/00; A61M 39/22

(52) U.S. Cl. CPC A61M 1/282; A61M 1/80; A61M 2039/229; A61M 2205/3337

(57) ABSTRACT

A peritoneal dialysis system includes a cycler having a pneumatic valve manifold or pneumatic arrangement, an air pump positioned and arranged to supply pneumatic pressure to the pneumatic valve manifold with or without intervening pneumatic storage, a pneumatic pressure sensor positioned and arranged to detect pneumatic pressure, and a control unit configured to use an output of the pressure sensor as feedback to adjust the air pump according to a set pneumatic pressure; and a disposable set including a pod pump having a flexible sheet, one side of the flexible sheet positioned and arranged during operation to receive pneumatic pressure via the air pump and pneumatic valve manifold.

### CROSS-REFERENCE TO RELATED APPLICATION

The present disclosure claims priority to Indian Provisional Patent Application 2024/41044759 having a filing date of Jun. 10, 2024, the entirety of which is incorporated herein.

### BACKGROUND

The present disclosure relates generally to medical fluid treatments and in particular to dialysis fluid treatments.

Due to various causes, a person's renal system can fail. Renal failure produces several physiological derangements. It is no longer possible to balance water and minerals or to excrete daily metabolic load. Toxic end products of metabolism, such as, urea, creatinine, uric acid and others, may accumulate in a patient's blood and tissue.

Reduced kidney function and, above all, kidney failure is treated with dialysis. Dialysis removes waste, toxins, and excess water from the body that normal functioning kidneys would otherwise remove. Dialysis treatment for replacement of kidney functions is critical to many people because the treatment is lifesaving.

One type of kidney failure therapy is Hemodialysis (“HD”), which in general uses diffusion to remove waste products from a patient's blood. A diffusive gradient occurs across the semi-permeable dialyzer between the blood and an electrolyte solution called dialysate or dialysis fluid to cause diffusion.

Hemofiltration (“HF”) is an alternative renal replacement therapy that relies on a convective transport of toxins from the patient's blood. HF is accomplished by adding substitution or replacement fluid to the extracorporeal circuit during treatment. The substitution fluid and the fluid accumulated by the patient in between treatments is ultrafiltered over the course of the HF treatment, providing a convective transport mechanism that is particularly beneficial in removing middle and large molecules.

Hemodiafiltration (“HDF”) is a treatment modality that combines convective and diffusive clearances. HDF uses dialysis fluid flowing through a dialyzer, similar to standard hemodialysis, to provide diffusive clearance. In addition, substitution solution is provided directly to the extracorporeal circuit, providing convective clearance.

Most HD, HF, and HDF treatments occur in centers. A trend towards home hemodialysis (“HHD”) exists today in part because HHD can be performed daily, offering therapeutic benefits over in-center hemodialysis treatments, which occur typically bi- or tri-weekly. Studies have shown that more frequent treatments remove more toxins and waste products and render less interdialytic fluid overload than a patient receiving less frequent but perhaps longer treatments. A patient receiving more frequent treatments does not experience as much of a down cycle (swings in fluids and toxins) as does an in-center patient, who has built-up two- or three-days' worth of toxins prior to a treatment. In certain areas, the closest dialysis center can be many miles from the patient's home, causing door-to-door treatment time to consume a large portion of the day. Treatments in centers close to the patient's home may also consume a large portion of the patient's day. HHD can take place overnight or during the day while the patient relaxes, works or is otherwise productive.

Another type of kidney failure therapy is peritoneal dialysis (“PD”), which infuses a dialysis solution, also called dialysis fluid, into a patient's peritoneal chamber via a catheter. The dialysis fluid is in contact with the peritoneal membrane in the patient's peritoneal chamber. Waste, toxins, and excess water pass from the patient's bloodstream, through the capillaries in the peritoneal membrane, and into the dialysis fluid due to diffusion and osmosis, i.e., an osmotic gradient occurs across the membrane. An osmotic agent in the PD dialysis fluid provides the osmotic gradient. Used or spent dialysis fluid is drained from the patient, removing waste, toxins, and excess water from the patient. This cycle is repeated, e.g., multiple times.

There are various types of peritoneal dialysis therapies, including continuous ambulatory peritoneal dialysis (“CAPD”), automated peritoneal dialysis (“APD”), tidal flow dialysis and continuous flow peritoneal dialysis (“CFPD”). CAPD is a manual dialysis treatment. Here, the patient manually connects an implanted catheter to a drain to allow used or spent dialysis fluid to drain from the peritoneal chamber. The patient then switches fluid communication so that the patient catheter communicates with a bag of fresh dialysis fluid to infuse the fresh dialysis fluid through the catheter and into the patient. The patient disconnects the catheter from the fresh dialysis fluid bag and allows the dialysis fluid to dwell within the peritoneal chamber, wherein the transfer of waste, toxins, and excess water takes place. After a dwell period, the patient repeats the manual dialysis procedure, for example, four times per day. Manual peritoneal dialysis requires a significant amount of time and effort from the patient, leaving ample room for improvement.

Automated peritoneal dialysis (“APD”) is similar to CAPD in that the dialysis treatment includes drain, fill and dwell cycles. APD machines, however, perform the cycles automatically, typically while the patient sleeps. APD machines free patients from having to manually perform the treatment cycles and from having to transport supplies during the day. APD machines connect fluidly to an implanted catheter, to a source or bag of fresh dialysis fluid and to a fluid drain. APD machines pump fresh dialysis fluid from a dialysis fluid source, through the catheter and into the patient's peritoneal chamber. APD machines also allow for the dialysis fluid to dwell within the chamber and for the transfer of waste, toxins, and excess water to take place. The source may include multiple liters of dialysis fluid including several solution bags.

APD machines pump used or spent dialysate from the peritoneal chamber, though the catheter, and to the drain. As with the manual process, several drain, fill and dwell cycles occur during dialysis. A “last fill” may occur at the end of the APD treatment. The last fill fluid may remain in the peritoneal chamber of the patient until the start of the next treatment or may be manually emptied at some point during the day.

In any of the above modalities using an automated machine, the automated machine operates typically with a disposable set, which is discarded after a single use. Depending on the complexity of the disposable set, the cost of using a set per day may become significant. Also, daily disposables require space for storage, which can become a nuisance for homeowners and businesses. Moreover, daily disposable replacement requires daily setup time and effort by the patient or caregiver at home or at a clinic. There is also a need for APD devices to be portable so that a patient may bring his or her device on vacation or for work travel.

For each of the above reasons, it is desirable to provide a relatively simple, compact APD machine, which operates a simple and cost-effective disposable set.

### SUMMARY

The present disclosure sets forth an automated peritoneal dialysis (“APD”) system having a machine or cycler that operates with a disposable set having a pod pump. In one possible configuration for the system, the disposable set includes multiple peritoneal dialysis fluid containers or bags, wherein one of the containers is placed on top of the cycler, which includes a heating plate to heat dialysis fluid located originally in the container as well as dialysis fluid pumped to the container from a second or later container for a subsequent patient fill. In an alternative embodiment, the plate or batch heater is replaced with an inline heater, which heats fresh dialysis fluid as it flows through the patient line to the patient. The disposable pumping pod or pod pump may be oriented vertically as illustrated herein, wherein fluid tubes or lines run horizontally from the pumping pod. An air pump for driving the disposable pod pump and other reusable components herein is located within a housing of the cycler.

The air pump is configured to provide both positive and negative pressure air to the disposable pod pump via a pneumatic valve manifold. The pneumatic valve manifold may include four pneumatic valves, including positive and negative pneumatic valves located between the air pump and the pod pump and reference chamber valves located between the pod pump and a reference chamber (located within the housing), wherein the reference chamber is used for fluid volume determinations discussed herein. In an embodiment, the air pump includes a box-in-a-box noise reducing structure in which inner and out noise reducing or attenuating encloses are provided about a pneumatic pump body to significantly reduce perceptible audible noise outputted by the air pump.

In an embodiment, a first pneumatic pressure sensor is located between the pneumatic valve manifold or pneumatic arrangement and the disposable pumping pod (pod pressure sensor). A second pneumatic pressure sensor is located between the pneumatic valve manifold or pneumatic arrangement and the reference chamber (reference pressure sensor). The pod and reference pressure sensors are used for the fluid volume determinations discussed herein. The pod pressure sensor is also used to control pumping pressure and to determine an end of stroke for the drawing and discharging of dialysis fluid into and from the pod pump.

The disposable set may include five fluid lines that extend from the disposable pumping pod, including a drain line that extends to a house drain (toilet, sink, or bathtub) or to a drain bag. Three peritoneal dialysis fluid containers or bags are provided in one embodiment, one of which sits atop a batch heater as mentioned above. The fifth line is a patient line. The disposable pumping pod mounted vertically to the front or actuation surface of the dialysis cycler, allows the drain line to be located at the top of the pumping pod and the patient line to be located at the bottom of the pumping pod. Such arrangement allows for air in the pod pump to migrate naturally upwardly into the drain line where it can be pumped to drain.

Each of the five fluid lines is fitted into or operates with a pinch valve, which may be an electrically actuated solenoid valve. The pinch valves are failsafe in one embodiment, meaning that upon power loss the valves are biased to close their respective fluid lines. The pinch valves alternatively retain their state upon power loss but are still part of a failsafe design in cooperation with the pod pump being deactivated upon power loss.

The pod pump may be constructed in multiple ways. In one embodiment the pod pump includes a rigid, e.g., plastic disposable shell and a flexible sheet, diaphragm or membrane fixed, e.g., ultrasonically welded, to the shell. Here, positive and negative pneumatic pressure is supplied from the air pump and the pneumatic valve manifold to the flexible sheet, diaphragm or membrane. In another embodiment, the pod pump includes two rigid plastic shells, namely, a pneumatic rigid plastic shell and a fluid contacting rigid plastic shell, which are sealed together to hold a flexible sheet, diaphragm or membrane in a sealed manner therebetween. A central pneumatic port is provided in the pneumatic rigid shell, which communicates pneumatically with the air pump and the pneumatic valve manifold. Here, air resides between the rigid plastic shell having the pneumatic port and the flexible membrane. In either of the above embodiments, five fluid ports extend from the fluid contacting rigid plastic shell, which connect to the five fluid lines discussed above. Fresh, fresh heated, or used dialysis fluid resides accordingly between the fluid contacting rigid plastic shell and the flexible membrane. In either embodiment, the flexible membrane is (i) pulled towards the actuation surface under negative pressure to pull fresh or used dialysis fluid into the disposable pumping pod, and (ii) pushed away from the actuation surface under positive pressure to push fresh or used dialysis fluid from the disposable pumping pod.

Other pod pump configurations include molded shells with reinforcement ribs with a dedicated rigid manifold, disposable pod pump(s) with a stopcock handle that are configured to selectively open one of a plurality of fluid ports while keeping other fluid ports closes, and a disposable pod pump with multiport stopcock valves.

The pod pump, the flexible plastic sheet, the fluid lines and fluid containers of the disposable set may be made of one or more plastic, e.g., polyvinylchloride (“PVC”) or a non-PVC material, such as polyethylene (“PE”), polyurethane (“PU”) or polycarbonate (“PC”). The housing of the cycler may be made of any of the above plastics, and/or of metal, e.g., stainless steel, steel and/or aluminum. As illustrated herein, the housing of the cycler may take different forms, e.g., the user interface may rotate up or out from the housing or may be integrated with the housing. A lid of the housing may be provided in halves that rotate outwardly to accept portions of a dialysis fluid/heater container or bag. Such arrangement allows an overall size and footprint to be smaller and to not be constrained at least in two dimensions by the size of the fluid/heater container or bag.

A control unit having one or more processor, one or more memory and a video controller operating with a user interface is provided to control each of the fluid valves, each of the pneumatic valves, the air pump, and the heater and to receive signals from each of the pressure sensors, the priming sensor or air detector, and one or more temperature sensor associated with the batch or inline the heater. The user interface may be provided with a touchscreen and/or electromechanical pushbuttons to allow the user or patient to enter parameters for treatment and a display screen for providing information, such as treatment status information. The control unit is also programmed to perform calculations based on the ideal gas law to determine how much fresh or used dialysis fluid has been pumped by the pod pump.

In one embodiment, the control unit is programmed to cause fresh or used dialysis fluid to be drawn into the pod pump using the following procedure. Here, the air pump is configured to be in a negative pressure or suction mode and is placed in pneumatic communication with the disposable pod pump via the opening of the negative pneumatic valve located between the air pump and the pod pump. A desired fluid source valve from which fluid is to be drawn into the disposable pumping pod is opened, e.g., a fresh dialysis fluid source valve, the heater bag valve or the patient valve. The control unit uses pressure feedback from the pod pressure sensor in an algorithm, e.g., a proportional, integral, derivative (“PID”) routine, to regulate the air pump to maintain a desired negative fluid pressure while fluid is pulled into the disposable pumping pod. In an embodiment, the control unit controls current to the air pump to adjust its speed and thus its negative pneumatic pressure output. The desired pressure may be different depending on the fluid source, e.g., −1.5 psig to-3.0 psig for pulling effluent from the patient or higher for pulling fresh PD fluid from a dialysis fluid container. The control unit in one embodiment uses a second algorithm to sense a spike in negative pressure and/or a corresponding drop in air pump speed to indicate an end of stroke and that the pod pump is filled with fresh or used dialysis fluid, which causes a trigger to stop the air pump.

In one embodiment, the control unit is programmed to measure an initial volume of fresh or used dialysis fluid drawn between the flexible sheet and the fluid contacting rigid plastic shell using two sets of pressure measurements and the ideal gas law. In a first set of pressure measurements, the control unit takes the pressure measurements of (i) the air side of the disposable pumping pod using the pod pressure sensor and (ii) the reference chamber using the reference pressure sensor. After fresh or used dialysis fluid is drawn into the pod pump, the control unit in a second set of pressure measurements opens one or more pneumatic valve(s) to allow the air side of the disposable pumping pod and the reference chamber to communicate pneumatically. Here, both the pod and reference pressure sensors measure the pressure of the combined cavity. Then, with all values on the right side of the following equation (e.g., “Equation 1”) known or measured (the volume of the reference chamber is known), the control unit calculates the volume of fluid pulled into the disposable pumping pod is as follows:

$${V}_{fluid initial} = {V}_{reference chamber} * ( {P}_{ref final} - {P}_{ref initial} ) / ( {P}_{pump initial} - {P}_{pump final} ) \qquad ( Eq . 1 )$$

In one embodiment, the control unit is programmed to cause fresh or used dialysis fluid to be pumped from the pod pump using the following procedure. Here, the air pump is configured to be in a positive pressure mode, which is placed in pneumatic communication with the disposable pumping pod via the opening of the positive pneumatic valve located between the air pump and the pod pump. A desired fluid destination valve through which fluid is to be delivered from the disposable pumping pod is opened, e.g., the heater bag valve, the patient valve or the drain valve. The control unit uses pressure feedback from the pod pressure sensor in the pressure algorithm, e.g., PID routine, to regulate the air pump to maintain a desired positive fluid pressure while fluid is discharged from the disposable pumping pod. Again, the control unit controls current to the air pump to adjust its speed and thus its positive pneumatic pressure output. The desired pressure may be different depending on the fluid destination, e.g., 3.0 psig to 8.0 psig for pushing fresh, heated dialysis fluid to the patient or higher for pushing to drain or the heating container.

The control unit in one embodiment uses an additional algorithm to sense a spike in positive pressure and/or a corresponding drop in air pump speed to indicate an end of stroke and that the pod pump has been emptied of fresh or used dialysis fluid, which causes a trigger to stop the air pump.

In one embodiment, the control unit is programmed to measure a final volume of fresh or used dialysis fluid located between the flexible sheet and the fluid contacting rigid plastic shell using the same two sets of pressure measurements and the ideal gas law. In a first set of pressure measurements, the control unit takes the pressure measurements of (i) the air side of the disposable pumping pod using the pod pressure sensor and (ii) the reference chamber using the reference pressure sensor. After fresh or used dialysis fluid is pumped from the pod pump, the control unit in a second set of pressure measurements opens one or more pneumatic valve(s) to allow the air side of the disposable pumping pod and the reference chamber to communicate pneumatically. Here, both the pod and reference pressure sensors measure the pressure of the combined cavity. Then, with all values on the right side of the following equation (e.g., “Equation 2”) known or measured, the control unit calculates the volume of fluid remaining in the pumping pod after the discharge stroke as follows:

$${V}_{fluid final} = {V}_{reference chamber} * ( {P}_{ref final} - {P}_{ref initial} ) / ( {P}_{pump initial} - {P}_{pump final} ) \qquad ( Eq . 2 )$$

The control unit then calculates the volume of fluid pumped from the pod pump by calculating the difference between the calculated pumping chamber volume before (V_{fluid initial}) and after (V_{fluid final}) pumping. The above steps or procedures are repeated until a required or prescribed volume of fresh or used dialysis fluid is pumped. The same pumping regime just described is used to pump fluid from any fluid source to any destination.

In light of the disclosure set forth herein, and without limiting the disclosure in any way, in a first aspect of the present disclosure, which may be combined with any other aspect, or portion thereof, a peritoneal dialysis system includes a cycler and a disposable set. The cycler includes a pneumatic valve manifold, an air pump positioned and arranged to supply positive and negative pneumatic pressure to the pneumatic valve manifold without intervening pneumatic storage, at least one pneumatic pressure sensor positioned and arranged to detect pneumatic pressure, and a control unit configured to use an output of the pneumatic pressure sensor as feedback to adjust the air pump according to a set pneumatic pressure. The disposable set includes a pod pump that has a flexible sheet attached to at least one pump housing. One side of the flexible sheet is positioned and arranged during operation to receive pneumatic pressure via the air pump and pneumatic valve manifold.

In another aspect of the present disclosure, which may be combined with any other aspect, or portion thereof, the flexible sheet is attached to a single pump housing.

In another aspect of the present disclosure, which may be combined with any other aspect, or portion thereof, the single pump housing includes a drain port, at least one dialysis fluid port, and a patient port.

In another aspect of the present disclosure, which may be combined with any other aspect, or portion thereof, the at least one pump housing includes a first pump housing and a second pump housing. The flexible sheet is positioned and arranged between the first pump housing and the second pump housing.

In another aspect of the present disclosure, which may be combined with any other aspect, or portion thereof, at least one of the first pump housing and the second pump housing includes a drain port, at least one dialysis fluid port, and a patient port.

In another aspect of the present disclosure, which may be combined with any other aspect, or portion thereof, the first pump housing has an internal surface facing the flexible sheet and an external surface, the external surface includes a first plurality of reinforcement ribs extending along the external surface in a first direction and a second plurality of reinforcement ribs extending along the external surface in a second direction.

In another aspect of the present disclosure, which may be combined with any other aspect, or portion thereof, the second pump housing has an internal surface facing the flexible sheet and an external surface, and the external surface includes a plurality of reinforcement ribs extending along the external surface.

In another aspect of the present disclosure, which may be combined with any other aspect, or portion thereof, the pneumatic valve manifold includes a plurality of valves, and the plurality of valves are at least one of pinch valves, stopcock valves, and volcano valves.

In another aspect of the present disclosure, which may be combined with any other aspect, or portion thereof, the disposable set includes a plurality of ports in fluid communication with the pod pump, the at least one pump housing includes a first pump housing and a second pump housing, and the second pump housing includes a stopcock handle configured to place a first port of the plurality of ports in an open arrangement while placing each of the other ports of the plurality of ports in a closed arrangement.

In another aspect of the present disclosure, which may be combined with any other aspect, or portion thereof, the cycler further includes a stepper motor configured to control a position of the stopcock handle. The position of the stopcock handle determines which port of the plurality of ports is in the open arrangement while each of the other ports of the plurality of ports is in the closed arrangement.

In another aspect of the present disclosure, which may be combined with any other aspect, or portion thereof, the plurality of ports includes a drain port, at least one supply port, and a patient port.

In another aspect of the present disclosure, which may be combined with any other aspect, or portion thereof, the pod pump includes a housing that has a plurality of multi-function combination ports.

In another aspect of the present disclosure, which may be combined with any other aspect, or portion thereof, the plurality of multi-function combination ports includes at least one of a first combination port configured to fluidly communicate with a drain port and heater port, a second combination port configured to fluidly communicate with a first supply port and second supply port, and a third combination drain port configured to fluidly communicate with a third supply port and a patient port.

In another aspect of the present disclosure, which may be combined with any other aspect, or portion thereof, the plurality of multi-function combination ports includes a first combination port configured to selectively actuate communication between a drain port and at least one of a heater port, a drain port, and a patient port.

In another aspect of the present disclosure, which may be combined with any other aspect, or portion thereof, the plurality of multi-function combination ports includes a first combination port configured to selectively actuate communication between a supply port and at least one of a heater port, an additional supply port, a drain port, and a patient port.

In another aspect of the present disclosure, which may be combined with any other aspect, or portion thereof, the plurality of multi-function combination ports includes a first combination port configured to selectively actuate communication between a drain port and at least one of a heater port, a supply port, and a patient port.

In light of the disclosure set forth herein, and without limiting the disclosure in any way, in a second aspect of the present disclosure, which may be combined with any other aspect, or portion thereof, a peritoneal dialysis system includes a cycler and a disposable set. The cycler includes a pneumatic valve manifold, an air pump positioned and arranged to supply positive and negative pneumatic pressure to the pneumatic valve manifold without intervening pneumatic storage, at least one pneumatic pressure sensor positioned and arranged to detect pneumatic pressure, and a control unit. The control unit is configured to use an output of the pneumatic pressure sensor as feedback to adjust the air pump according to a set pneumatic pressure. The disposable set includes a pod pump, a plurality of ports and a stopcock handle configured to selectively open and close one or more ports of the plurality of ports.

In another aspect of the present disclosure, which may be combined with any other aspect, or portion thereof, the plurality of ports includes a drain port, at least one dialysis fluid port, and a patient port.

In light of the disclosure set forth herein, and without limiting the disclosure in any way, in a third aspect of the present disclosure, which may be combined with any other aspect, or portion thereof, a peritoneal dialysis system includes a cycler and a disposable set. The cycler includes a pneumatic valve manifold, an air pump positioned and arranged to supply positive and negative pneumatic pressure to the pneumatic valve manifold without intervening pneumatic storage, at least one pneumatic pressure sensor positioned and arranged to detect pneumatic pressure, and a control unit configured to use an output of the pneumatic pressure sensor as feedback to adjust the air pump according to a set pneumatic pressure. The disposable set includes a pod pump that has a flexible sheet and a plurality of multi-function combination ports. One side of the flexible sheet is positioned and arranged during operation to receive pneumatic pressure via the air pump and pneumatic valve manifold.

In another aspect of the present disclosure, which may be combined with any other aspect, or portion thereof, the plurality of multi-function combination ports includes a first combination port configured to fluidly communicate with a drain port and heater port, a second combination port configured to fluidly communicate with a first supply port and second supply port, and a third combination drain port configured to fluidly communicate with a third supply port and a patient port.

In light of the disclosure set forth herein, and without limiting the disclosure in any way, in a fourth aspect of the present disclosure, which may be combined with any other aspect, or portion thereof, a peritoneal dialysis system includes a cycler, a reference chamber, and a disposable set. The cycler includes a pneumatic valve manifold that has a plurality of pneumatic valves, an air pump positioned and arranged to supply positive and negative pneumatic pressure to the pneumatic valve manifold without intervening pneumatic storage, at least one pneumatic pressure sensor positioned and arranged to detect pneumatic pressure, and a control unit configured to use an output of the pneumatic pressure sensor as feedback to adjust the air pump according to a set pneumatic pressure. The disposable set includes a pod pump that has a flexible sheet. One side of the flexible sheet is positioned and arranged during operation to receive pneumatic pressure via the air pump and pneumatic valve manifold.

In another aspect of the present disclosure, which may be combined with any other aspect, or portion thereof, the plurality of pneumatic valves includes a first pneumatic valve positioned and arranged to allow the reference chamber to communicate pneumatically with the side of the flexible sheet positioned and arranged during operation to receive pneumatic pressure.

In another aspect of the present disclosure, which may be combined with any other aspect, or portion thereof, the air pump is configured to supply positive and negative pneumatic pressure.

In another aspect of the present disclosure, which may be combined with any other aspect, or portion thereof, the plurality of pneumatic valves includes, a positive pneumatic valve, a negative pneumatic valve and at least one additional pneumatic valve, the control unit is further configured to use outputs from the at least one pressure sensor in combination with a sequence of the positive pneumatic valve, negative pneumatic valve and the at least one additional pneumatic valve and an ideal gas law equation to compute a fluid volume.

In another aspect of the present disclosure, which may be combined with any other aspect, or portion thereof, the fluid volume is at least one of (i) an initial dialysis fluid volume in the pod pump after fresh or used dialysis fluid is drawn into the pod pump by supplying a positive pressure to the reference chamber and later venting the reference chamber and (ii) a final dialysis fluid volume in the pod pump after fresh or used dialysis fluid is pumped from the pod pump by supplying a negative pressure to the reference chamber and later venting the reference chamber.

In another aspect of the present disclosure, which may be combined with any other aspect, or portion thereof, the control unit is further configured to subtract (ii) from (i) to determine a volume of the fresh or used dialysis fluid pumped from the pod pump.

In another aspect of the present disclosure, which may be combined with any other aspect, or portion thereof, the reference chamber is a first reference chamber and the peritoneal dialysis system further includes a second reference chamber. The air pump is configured to supply positive and negative pneumatic pressure. The pneumatic valve manifold further includes at least one additional pneumatic valve positioned and arranged to allow the first reference chamber and the second reference chamber to communicate pneumatically with the side of the flexible sheet positioned and arranged during operation to receive pneumatic pressure.

In another aspect of the present disclosure, which may be combined with any other aspect, or portion thereof, the control unit is further configured to alternate between the first reference chamber and the second reference chamber to (i) perform fluid volume determinations and (ii) supply positive and negative pneumatic pressure to the one side of the flexible sheet.

In light of the disclosure set forth herein, and without limiting the disclosure in any way, in a fifth aspect of the present disclosure, which may be combined with any other aspect, or portion thereof, a peritoneal dialysis system includes a disposable set and a cycler. The disposable set includes a pod pump that has a flexible sheet with one side of the flexible sheet positioned and arranged during operation to receive pneumatic pressure. The cycler includes a pneumatic valve manifold for providing pneumatic pressure to the side of the flexible sheet positioned and arranged during operation to receive pneumatic pressure, an air pump positioned and arranged to supply pneumatic pressure to the pneumatic valve manifold, at least one reference chamber in pneumatic communication with the pneumatic valve manifold, a plurality of pressure sensors, and a control unit. The control unit is configured to use outputs from at least one pressure sensor of the plurality of pressure sensors in combination with a sequence of the pneumatic valve manifold and an ideal gas law equation to compute at least one of (i) an initial dialysis fluid volume in the pod pump after fresh or used dialysis fluid is drawn into the pod pump or (ii) a final dialysis fluid volume in the pod pump after fresh or used dialysis fluid is pumped from the pod pump.

In another aspect of the present disclosure, which may be combined with any other aspect, or portion thereof, the control unit is further configured to subtract (ii) from (i) to determine a volume of the fresh or used dialysis fluid pumped from the pod pump.

In another aspect of the present disclosure, which may be combined with any other aspect, or portion thereof, the control unit is further configured to repeat (i) and (ii) and to subtract (ii) from (i) until a prescribed volume of fresh or used dialysis fluid is pumped.

In another aspect of the present disclosure, which may be combined with any other aspect, or portion thereof, the at least one reference chamber includes a first reference chamber and a second reference chamber. The air pump is positioned and arranged to supply pneumatic pressure to the pneumatic valve manifold without intervening pneumatic storage.

In another aspect of the present disclosure, which may be combined with any other aspect, or portion thereof, the peritoneal dialysis further includes a first dedicated pressure tank and a second dedicated pressure tank. The first dedicated pressure tank is positioned and arranged to pneumatically store positive pneumatic pressure, and the second dedicated pressure tank is positioned and arranged to pneumatically store negative pneumatic pressure.

In light of the disclosure set forth herein, and without limiting the disclosure in any way, in a fifth aspect of the present disclosure, which may be combined with any other aspect, or portion thereof, a method includes creating negative pressure on a pump chamber and positive pressure on a reference chamber and maintaining the pump chamber at a first pressure and the reference chamber at a second pressure. The method also includes placing the pump chamber and the reference chamber in fluid communication, at a first time, to transition the pump chamber and the reference chamber to a transition pressure. Additionally, the method includes venting the reference chamber and performing a first volume calculation.

In another aspect of the present disclosure, which may be combined with any other aspect, or portion thereof, an air pump creates the negative pressure on the pump chamber and the positive pressure on the reference chamber in conjunction with at least one valve of a plurality of pneumatic valves of a pneumatic valve manifold. Placing the pump chamber and the reference chamber in fluid communication includes opening a first valve of the plurality of pneumatic valves of the pneumatic valve manifold. The first valve is positioned downstream of the air pump and along a pneumatic line connected to both the pump chamber and the reference chamber.

In another aspect of the present disclosure, which may be combined with any other aspect, or portion thereof, venting the reference chamber includes placing the reference chamber in fluid communication with at least one valve of a plurality of pneumatic valves of a pneumatic valve manifold. The at least one valve includes a vent, and the reference chamber is vented via the vent.

In another aspect of the present disclosure, which may be combined with any other aspect, or portion thereof, the transition pressure is greater than the first pressure and less than the second pressure.

In another aspect of the present disclosure, which may be combined with any other aspect, or portion thereof, the method further includes maintaining the pump chamber at a third pressure and building a negative pressure in the reference chamber. The method also includes placing the reference chamber and the pump chamber in fluid communication, at a second time, to adjust the pressure in the pump chamber to a fourth pressure. Additionally, the method includes performing a second volume calculation.

In another aspect of the present disclosure, which may be combined with any other aspect, or portion thereof, the method further includes subtracting the second volume calculation from the first volume calculation.

In another aspect of the present disclosure, which may be combined with any other aspect, or portion thereof, the method is performed by a control unit of a cycler. The control unit configured to use an output of a pneumatic pressure sensor of the cycler as feedback to adjust an air pump of the cycler according to a set pneumatic pressure.

In light of the disclosure set forth herein, and without limiting the disclosure in any way, in a sixth aspect of the present disclosure, which may be combined with any other aspect, or portion thereof, a peritoneal dialysis system includes a disposable set and a cycler. The disposable set includes a pod pump that has a flexible sheet. One side of the flexible sheet positioned and arranged during operation to receive pneumatic pressure. The cycler includes a pneumatic valve manifold for providing pneumatic pressure to the side of the flexible sheet positioned and arranged during operation to receive pneumatic pressure. The cycler also includes an air pump positioned and arranged to supply pneumatic pressure to the pneumatic valve manifold, at least one reference chamber in pneumatic communication with the pneumatic valve manifold, and a plurality of pressure sensors. The cycler also includes a control unit. The control unit is configured to make a determination from a sensed speed of the air pump, the control unit is further configured to use outputs from at least one pressure sensor of the plurality of pressure sensors in combination with a sequence of the pneumatic valve manifold and an ideal gas law equation to compute at least one of (i) an initial dialysis fluid volume in the pod pump after fresh or used dialysis fluid is drawn into the pod pump or (ii) a final dialysis fluid volume in the pod pump after fresh or used dialysis fluid is pumped from the pod pump.

In another aspect of the present disclosure, which may be combined with any other aspect, or portion thereof, the control unit is further configured to determine that the flexible sheet has completed drawing fresh or used dialysis fluid into the pod pump based on the sensed speed of the air pump.

In another aspect of the present disclosure, which may be combined with any other aspect, or portion thereof, the control unit is further configured to determine that the flexible sheet has completed pumping fresh or used dialysis fluid from the pod pump based on the sensed speed of the air pump.

In another aspect of the present disclosure, which may be combined with any other aspect, or portion thereof, the control unit is further configured to determine that a drain operation is complete based on the sensed speed of the air pump.

In another aspect of the present disclosure, which may be combined with any other aspect, or portion thereof, the control unit is further configured to determine that an occlusion occurred based on the sensed speed of the air pump.

In another aspect of the present disclosure, which may be combined with any other aspect, or portion thereof, the cycler further includes a flow sensor positioned and arranged to measure a flow rate of air from the air pump.

In another aspect of the present disclosure, which may be combined with any other aspect, or portion thereof, the control unit is further configured to determine that the flexible sheet has completed drawing fresh or used dialysis fluid into the pod pump based on the measured flow rate of air.

In another aspect of the present disclosure, which may be combined with any other aspect, or portion thereof, the control unit is further configured to determine that the flexible sheet has completed pumping fresh or used dialysis fluid from the pod pump based on the measured flow rate of air.

In another aspect of the present disclosure, which may be combined with any other aspect, or portion thereof, the control unit is further configured to determine that a drain operation is complete based on the measured flow rate of air.

In another aspect of the present disclosure, which may be combined with any other aspect, or portion thereof, the control unit is further configured to determine that an occlusion occurred based on the measured flow rate of air.

In another aspect of the present disclosure, which may be combined with any other aspect, or portion thereof, the control unit is further configured to (i) determine that a drain operation is complete or (i) detect an occlusion condition based on the sensed speed of the air pump and the measured flow rate of air.

In another aspect of the present disclosure, which may be combined with any other aspect, or portion thereof, the control unit is configured to monitor the sensed speed of the air pump to determine that the sensed speed of the air pump has reached a predetermined threshold.

In another aspect of the present disclosure, which may be combined with any other aspect, or portion thereof, the control unit is configured to determine that a push back procedure is successful.

In another aspect of the present disclosure, which may be combined with any other aspect, or portion thereof, the control unit is configured to indicate that a drain operation is complete based on determining that the push back procedure is successful.

In another aspect of the present disclosure, which may be combined with any other aspect, or portion thereof, the control unit configured to (i) monitor a sensed speed of the air pump to determine that the sensed speed of the air pump has reached a predetermined threshold; (ii) determine that a push back procedure is unsuccessful; and (iii) based on determining that the push back procedure is unsuccessful, detect an occlusion condition.

In light of the disclosure set forth herein, and without limiting the disclosure in any way, in a seventh aspect of the present disclosure, which may be combined with any other aspect, or portion thereof, a method includes monitoring a sensed speed of the air pump, determining that the sensed speed of the air pump has reached a predetermined threshold, and determining a status of a push back procedure as one of (i) successful and (ii) unsuccessful. The method also includes indicating that a drain operation is complete responsive to determining the status of the push back procedure is successful. Additionally, the method includes detecting an occlusion condition responsive to determining the status of the push back procedure is unsuccessful.

In another aspect of the present disclosure, which may be combined with any other aspect, or portion thereof, monitoring the sensed speed of the air pump includes monitoring, by a control unit of a cycler of a peritoneal dialysis system, the sensed speed of the air pump.

In another aspect of the present disclosure, which may be combined with any other aspect, or portion thereof, determining a status of a push back procedure as one of (i) successful and (ii) unsuccessful includes determining, by the control unit, the status of the push back procedure.

In another aspect of the present disclosure, which may be combined with any other aspect, or portion thereof, the method further includes calculating, by the control unit, at least one of (i) an initial dialysis fluid volume in a pod pump of a disposable set of the peritoneal dialysis system and (ii) a final dialysis fluid volume in the pod pump after fresh or used dialysis fluid is pumped from the pod pump. The pod pump has a flexible sheet, one side of the flexible sheet is positioned and arranged during operation to receive pneumatic pressure.

In another aspect of the present disclosure, which may be combined with any other aspect, or portion thereof, the control unit performs calculations using an output from at least one pressure sensor of the cycler in combination with a sequence of a pneumatic valve manifold of the cycler and an ideal gas law equation.

In another aspect of the present disclosure, which may be combined with any other aspect, or portion thereof, any of the features, functionality and alternatives described in connection with any one or more of FIGS. 1A to 13 may be combined with any of the features, functionality and alternatives described in connection with any other of FIGS. 1A to 13.

In light of the above aspects and present disclosure, it is an advantage of the present disclosure to provide a relatively volumetrically accurate automated peritoneal dialysis (“APD”) cycler.

It is another advantage of the present disclosure to provide an APD cycler that achieves relatively precise pressure control.

It is still another advantage of the present disclosure to provide a pneumatically operated APD cycler that does not require pressure storage devices.

It is still a further advantage of the present disclosure to provide an APD system that is able to build motive fluid or pneumatic pressure in a relatively simple manner.

It is another advantage of the present disclosure to provide a simple and quick process for detecting an occlusion (e.g., kink) or “end of drain” to reduce therapy time and drain pain.

It is still another advantage of the present disclosure to provide higher flow rates (e.g., 10-15% gain over previous techniques), which advantageously reduces therapy time.

It is still another advantage of the present disclosure to provide a simple process, means or technique of measuring instantaneous flow rate.

It is yet another advantage of the present disclosure to provide an APD system that employs a relatively low-cost disposable set.

It is yet a further advantage of the present disclosure to provide an APD system that employs a disposable pod pump with matching shells having reinforcement ribs to reduce material while maintaining sufficient strength, which saves disposable cost.

Additional features and advantages are described in, and will be apparent from, the following Detailed Description and the Figures. The features and advantages described herein are not all-inclusive and, in particular, many additional features and advantages will be apparent to one of ordinary skill in the art in view of the figures and description. Also, any particular embodiment does not have to have all of the advantages listed herein and it is expressly contemplated to claim individual advantageous embodiments separately. Moreover, it should be noted that the language used in the specification has been selected principally for readability and instructional purposes, and not to limit the scope of the inventive subject matter.

### BRIEF DESCRIPTION OF THE FIGURES

FIG. 1A is a schematic view of an automated peritoneal dialysis (“APD”) cycler using a pod pump, according to an example embodiment of the present disclosure.

FIG. 1B is a perspective view of an APD cycler using a pod pump of the system, according to an example embodiment of the present disclosure.

FIGS. 2A-2D are schematic views of pneumatic arrangements for the APD cycler using a pod pump illustrated in FIG. 1A, according to an example embodiment of the present disclosure.

FIG. 3 is a process flow diagram for determining fresh or used dialysis fluid volume delivery, according to an example embodiment of the present disclosure.

FIGS. 4A-4B are schematic views of flow paths relating to the process flow diagram in FIG. 3, according to an example embodiment of the present disclosure.

FIG. 5 is a process flow diagram of drain logic for determining end of drain and occlusion detection, according to an example embodiment of the present disclosure.

FIG. 6 is a process flow diagram of closed loop pressure control for reaching and maintaining a target pressure, according to an example embodiment of the present disclosure.

FIG. 7A is a perspective view of a disposable pod pump, according to an example embodiment of the present disclosure.

FIG. 7B is an exploded perspective view of the disposable pod pump of FIG. 7A, according to an example embodiment of the present disclosure.

FIG. 7C is a cross-sectional view along line A-A of FIG. 7B of a fluid contacting shell of the pod pump of FIGS. 7A and 7B, according to an example embodiment of the present disclosure.

FIG. 8A is a sectioned elevation view of a pod pump with corresponding valve manifold and fluid lines, according to an example embodiment of the present disclosure.

FIG. 8B is a sectioned perspective view of the pod pump with corresponding valve manifold and fluid lines of FIG. 8A, according to an example embodiment of the present disclosure.

FIG. 9A is a front view of a disposable pod pump, according to an example embodiment of the present disclosure.

FIG. 9B is a side view of the disposable pod pump illustrated in FIG. 9A, according to an example embodiment of the present disclosure.

FIG. 10A is a front view of a disposable pod pump, according to an example embodiment of the present disclosure.

FIG. 10B is a side view of the disposable pod pump illustrated in FIG. 10A, according to an example embodiment of the present disclosure.

FIG. 11 is a front view of a disposable pod pump, according to an example embodiment of the present disclosure.

FIG. 12 is a plot of motor speed and pumping chamber pressure during a fluid management system cycle as described in relation to FIG. 3, according to an example of the present disclosure.

FIG. 13 is a plot of pump speed and pumping pressure when implementing the drain logic of FIG. 5, according to an example of the present disclosure.

### DETAILED DESCRIPTION

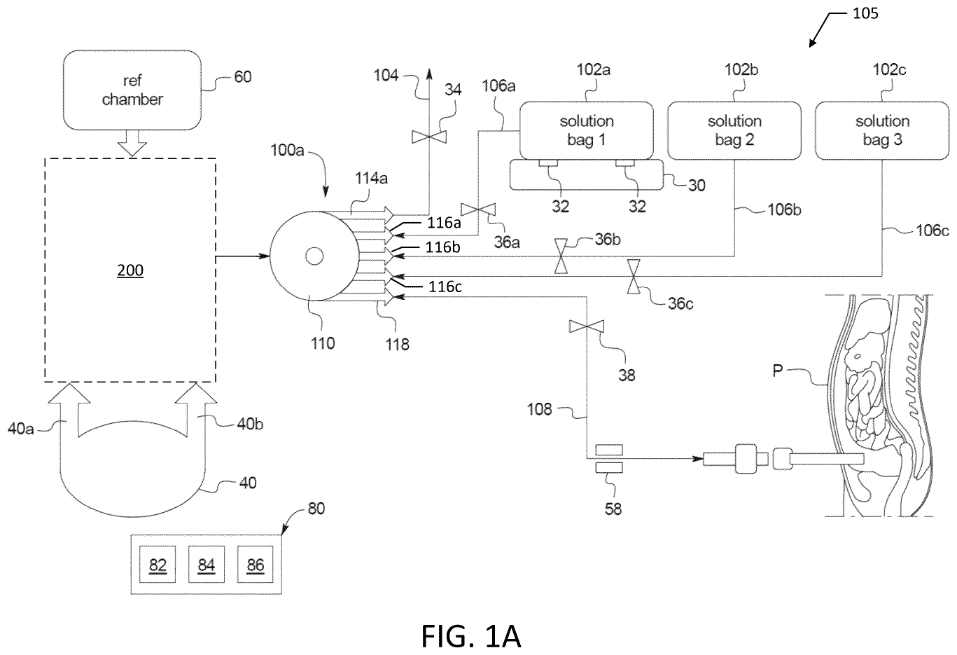

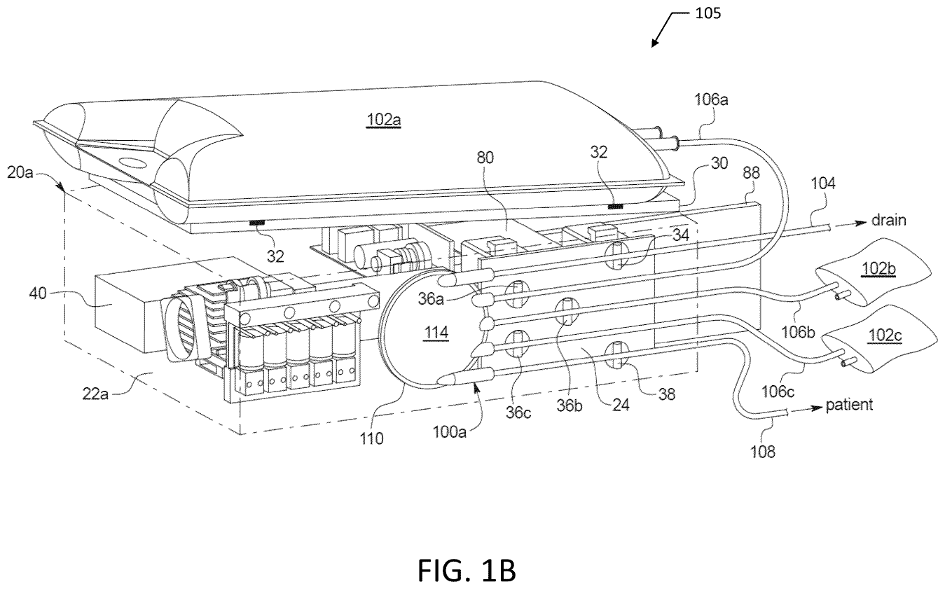

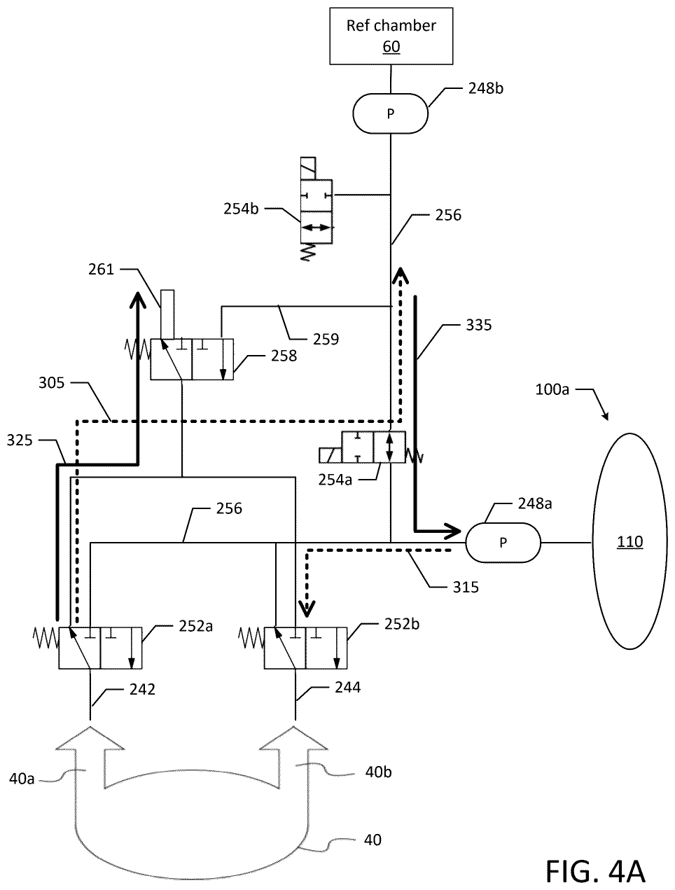

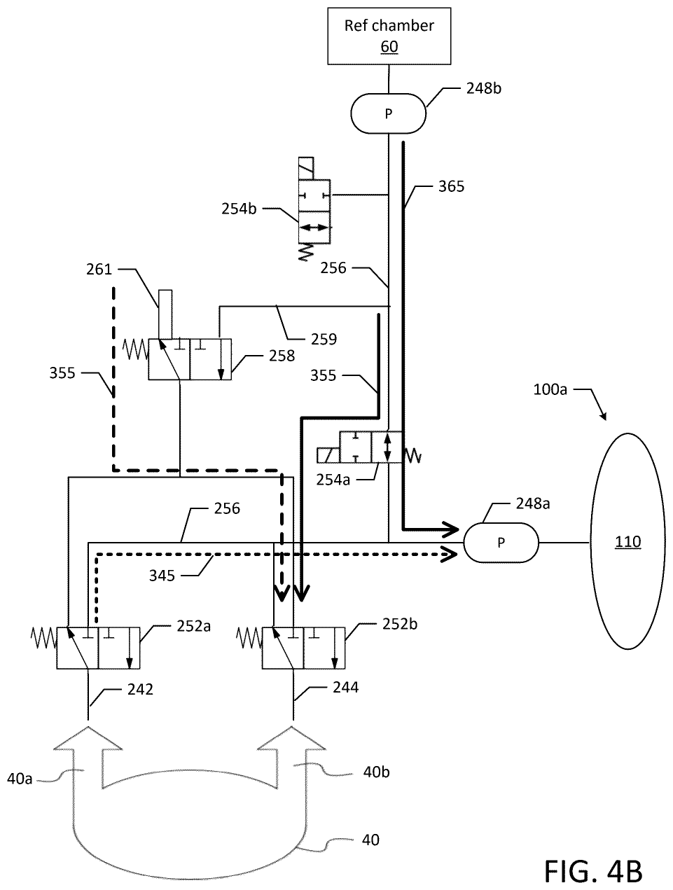

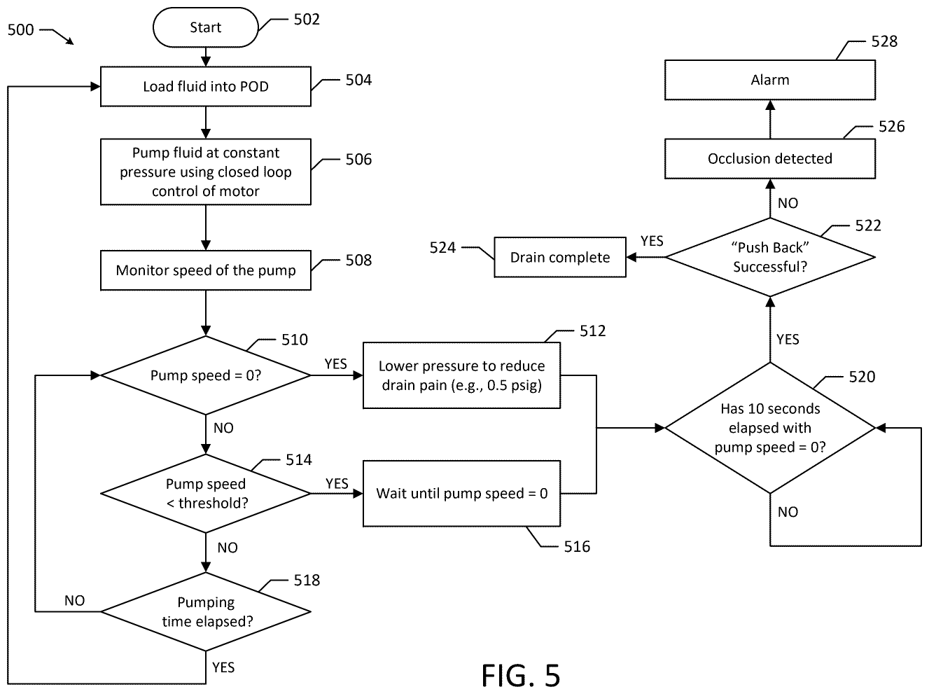

Referring now to the drawings and in particular to FIGS. 1A and 1B, an embodiment of system 105 includes an automated peritoneal dialysis (“APD”) cycler (e.g., cycler 20a having a housing 22a as illustrated in FIG. 1B), which operates with a disposable set 100a that organizes tubing and performs many functions discussed herein. Disposable set 100a includes a pod pump 110, which in one embodiment is covered on one side by a flexible sheet, diaphragm or membrane (e.g., plastic sheet 112 illustrated in FIGS. 7A and 7B) that is easy and cost effective to manufacture, e.g., so that two rigid plastic pieces do not have to be welded together.

As illustrated in FIG. 1B, disposable set 100a including pod pump 110 is inserted for operation inside of APD cycler 20a, for example, in between an actuation surface 24 and a door 26 of the APD cycler. Door 26, for example, hinges open via one or more hinge 28 located along a bottom of cycler housing 22a, adjacent to actuation surface 24. In the illustrated implementation, pod pump 110 of disposable set 100a is mounted so that the flexible sheet, diaphragm or membrane 112 of pod pump 110 when mounted for operation is located facing actuation surface 24 of cycler 20a. Door 26 when closed constrains pod pump 110 against actuation surface 24 during operation in one embodiment.

As illustrated in FIG. 1B, disposable set 100a including pod pump 110 is inserted for operation inside of APD cycler 20a, for example, in between an actuation surface 24 and a door (not pictured) of the APD cycler. The door, for example, may hinge open via one or more hinge(s) (not pictured) located along a bottom of cycler housing 22a, adjacent to actuation surface 24. In the illustrated implementation, pod pump 110 of disposable set 100a is mounted so that plastic sheet (e.g., plastic sheet 112, see FIGS. 7A, 7B) of pod pump 110 when mounted for operation is located facing actuation surface 24 of cycler 20a. The door, when closed, constrains pod pump 110 against actuation surface 24 during operation in one embodiment.

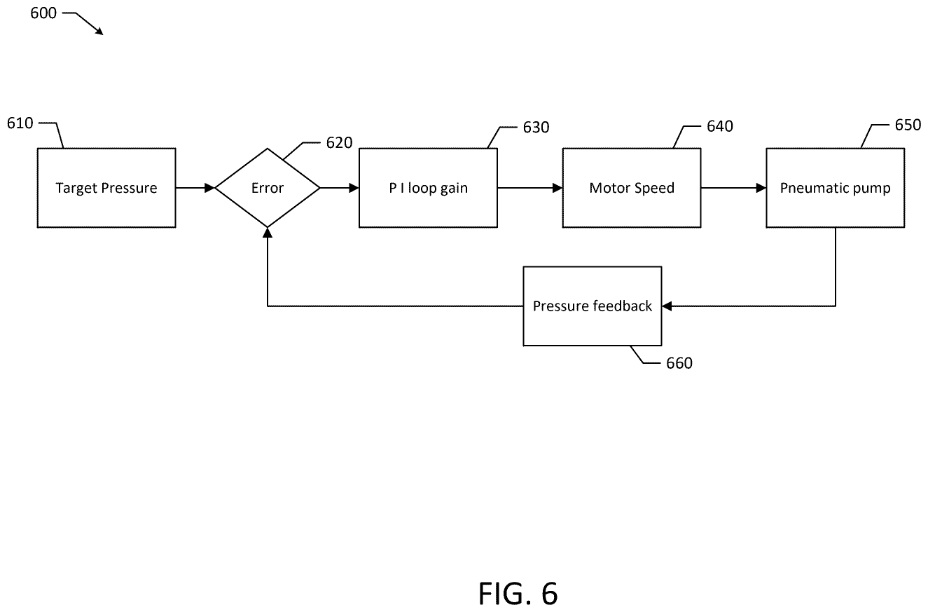

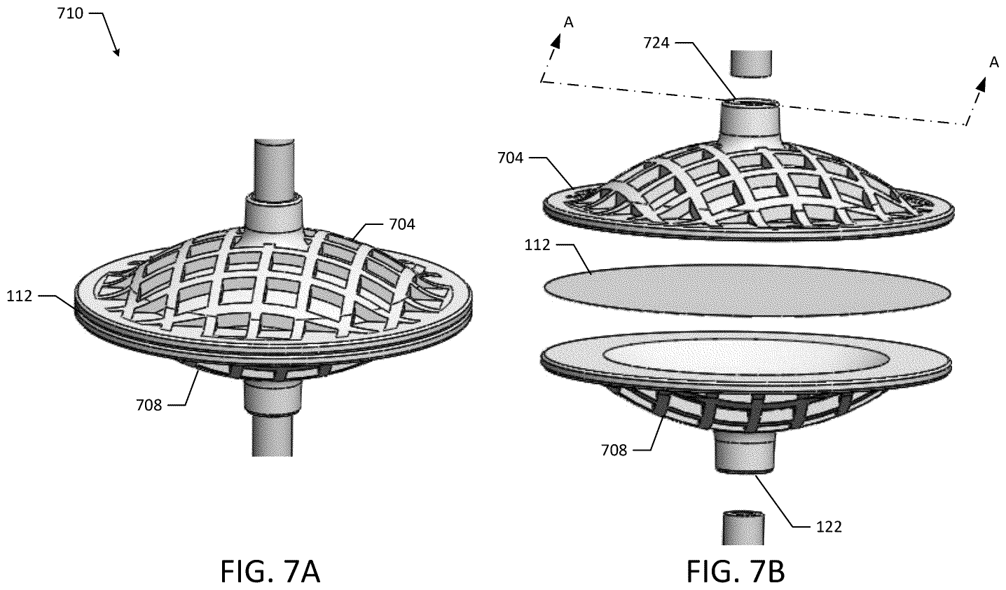

In one possible configuration for system 105 as illustrated in FIGS. 1A and 1B, disposable set 100a includes multiple peritoneal dialysis fluid containers or bags 102a to 102c, wherein one of the containers, e.g., container 102a, is placed on top of cycler 20a, which includes a dialysis fluid heater 30, which may be a plate or batch heater, to heat dialysis fluid located originally in container 102a as well as dialysis fluid pumped to container 102a from second and third containers 102b and 102c for subsequent patient fills. In an alternative embodiment, the dialysis fluid heater 30 (e.g., plate or batch heater) is replaced with an inline dialysis fluid heater (not illustrated here, see example of inline heater 830 illustrated in FIGS. 8A and 8B), which heats fresh dialysis fluid as it flows through a patient line to the patient. A serpentine inline heating pathway may be provided along the patient line to aid the inline heating (see example of inline heater 830 illustrated in FIGS. 8A and 8B). The serpentine inline heating pathway may be placed on top of cycler 20a or be inserted into the cycler for heating, e.g., via resistive plate heating.

Disposable pumping pod or pod pump 110 is oriented vertically in the illustrated embodiment of FIG. 1B, wherein fluid tubes or lines run horizontally from the pumping pod. In the illustrated embodiment, pod pump 110 includes a fluid contacting fluid contacting rigid plastic shell 114 to which a flexible sheet (e.g., flexible sheet 112) is attached, e.g., ultrasonically sealed. As illustrated in FIGS. 1A and 1B, pod pump 110 defines a drain port 114a that accepts a drain line 104 in a sealed manner, wherein drain line 104 extends to a drain container or house drain, e.g., toilet, bathtub or sink (not illustrated). Pod pump 110, or more specifically the fluid contacting rigid plastic shell 114, includes or defines a first dialysis fluid port 116a that accepts a first dialysis fluid line 106a in a sealed manner, wherein first dialysis fluid line 106a extends to fresh dialysis fluid container 102a positioned on the top of cycler 20a, which doubles as the fluid heating container. Pod pump 110, or more specifically the fluid contacting rigid plastic shell 114, includes or defines a second dialysis fluid port 116b that accepts a second dialysis fluid line 106b in a sealed manner, wherein second dialysis fluid line 106b extends to fresh dialysis fluid container 102b. Pod pump 110, or more specifically the fluid contacting rigid plastic shell 114, includes or defines a third dialysis fluid port 116c that accepts a third dialysis fluid line 106c in a sealed manner, wherein third dialysis fluid line 106c extends to fresh dialysis fluid container 102c. Pod pump 110, or more specifically the fluid contacting rigid plastic shell 114, includes or defines a patient port 118 that accepts a patient line 108 in a sealed manner, wherein patient line 108 extends to a patient connector (not illustrated) that connects to a patient's transfer set and communicates fluidly with the patient's indwelling peritoneal dialysis catheter.

It should be appreciated that the pod pump 110 may have different design(s) or construction(s) than the rigid plastic shell 114 illustrated in FIG. 1B, the additional examples and embodiments are described later in reference to FIGS. 7A, 7B, 7C, 8, 9A, 9B, 10A, 10B and 11.

Disposable set 100a may alternatively provide more or less than the illustrated five fluid lines, e.g., the number of fresh dialysis fluid containers may be varied. Fresh dialysis fluid containers 102a to 102c may contain the same or different types and volumes of fresh dialysis fluids. For example, fresh dialysis fluid containers 102a to 102c may contain different levels of dextrose or glucose. One of the containers may contain a different formulation of fresh dialysis fluid, e.g., icodextrin, for a patient's last fill.

With disposable set 100a and pumping pod 110 mounted vertically to actuation surface 24 of cycler 20a, drain line 104 is located at the top of the pumping pod, while patient line 108 is located at the bottom of the pumping pod. Such arrangement allows for air in pod pump 110 to migrate naturally upwardly into drain line 104 where it can be pumped to the house drain or drain container.

To load disposable set 100a and pod pump 110, the patient or user in one embodiment opens a door (not pictured) of the cycler and loads disposable set 100a into the inside of the door as the door lays flat or almost flat on a table, nightstand, desk, etc. The patient or caregiver then closes the door, causing pod pump 110 and fluid lines 104, 106a to 106c and patient line 108 to come into registry, respectively, with one or more pneumatic delivery aperture or port formed in an actuation surface and with pinch valves 34, 36a to 36c and 38 located along actuation surface 24. The patient or caregiver then loads fresh dialysis fluid container 102a onto resistive plate or batch dialysis fluid heater 30 assuming that batch heating is provided. Dialysis fluid containers or bags 102b and 102c may be located in a convenient position on the table, nightstand, desk, etc.

As illustrated in FIG. 1A, disposable set 100a, dialysis fluid heater 30 and pinch valves 34, 36a to 36c and 38 are illustrated along with other components of system 105. Dialysis fluid heater 30, priming/air sensor 58, and pinch valves 34, 36a to 36c and 38 and each of the components to the left of pod pump 110 in FIG. 1A may be durable, reusable components. Cycler 20a provides an air pump 40 for pneumatically driving disposable pod pump 110. Air pump 40 in one embodiment is bidirectional and can supply positive and negative pneumatic pressure, e.g., positive pneumatic pressure out of positive port 40a and negative pneumatic pressure out of negative port 40b.

Suitable air pumps 40 may for example be diaphragm pumps, scroll pumps or piston pumps. Air pump 40 may be electrically actuated such that the amount of current delivered to the air pump determines the speed of the pump and the pressure and flowrate of air delivered to pod pump 110.

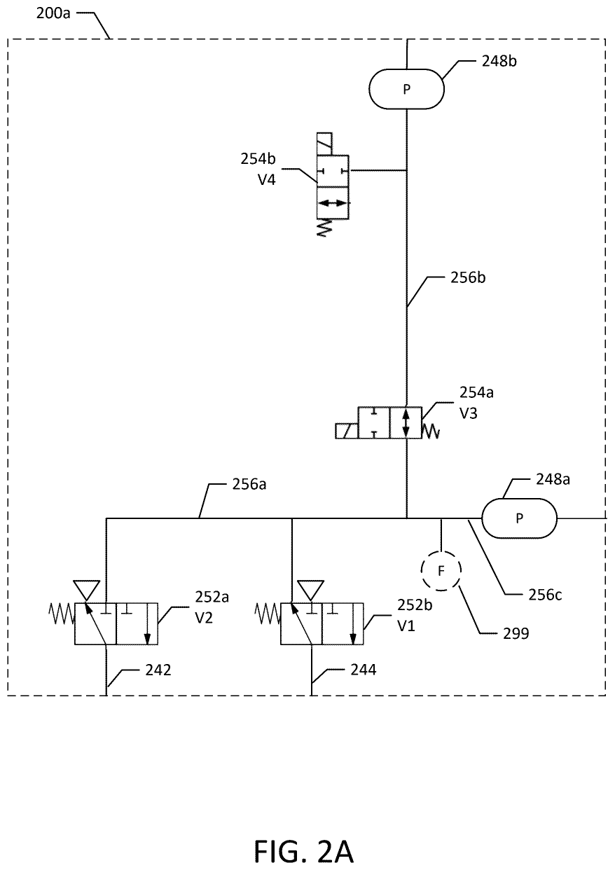

In the illustrated embodiment, air pump 40 delivers both positive and negative pressure air to disposable pod pump 110 via a pneumatic arrangement 200 that may include a pneumatic valve manifold. Various pneumatic arrangements 200a, 200b, 200c and 200d are illustrated in FIGS. 2A-2D.

FIGS. 1A and 1B also illustrates that each of the five fluid lines 104, 106a to 106c and patient line 108 is fitted into or operates respectively with a pinch valve 34, 36a to 36c and 38, which may each be an electrically actuated solenoid valve. Pinch valves 34, 36a to 36c and 38 are failsafe in one embodiment, meaning that upon power loss the valves are biased to close their respective fluid lines. The pinch valves alternatively retain their state upon power loss but are still part of a failsafe design in cooperation with pod pump 110 being deactivated upon power loss. In some embodiments, alternate valve mechanisms may be used in addition to or in place of the pinch valves. In such embodiments, the valves may be stopcock valves, volcano valves, or any other valves known in the art.

FIGS. 1A and 1B illustrate that cycler 20a includes a control unit 80 having one or more processor 82, one or more memory 84 and a video controller 86 operating with a user interface (not pictured) provided to control each of fluid valves 34, 36a to 36c and 38, each of pneumatic valves (e.g., pneumatic valves of FIGS. 2A-2D, such as pneumatic valves 252a, 252b, 254a and 254b of FIG. 2A), air pump 40, and heater 30 and to receive signals from each of the pressure sensors 248a, 248b, prime sensor/bubble detector (not pictured), and one or more temperature sensor 32 associated with batch or inline heater 30. Control unit 80 may use feedback from one or more temperature sensor 32 to adjust the amount of power delivered to heater 30, e.g., via a proportional, integral, derivative (“PID”) routine, so that heated, fresh dialysis fluid is delivered to the patient at a desired temperature, e.g., 37° C.

As illustrated in FIGS. 1A and 1B, to draw fresh dialysis fluid into pod pump 110, negative pressure is applied via air pump 40 and one of dialysis fluid pinch valves 36a to 36c is opened, while all other pinch valves are closed. To draw fresh, heated dialysis fluid into pod pump 110, negative pressure is applied and dialysis fluid/heater pinch valve 36a is opened, while all other pinch valves are closed. To draw used dialysis fluid from patient P into pod pump 110, negative pressure is applied and patient valve 38 is opened, while all other pinch valves are closed. To push fresh, heated dialysis fluid from pod pump 110 to patient P, positive pressure is applied and patient valve 38 is opened, while all other pinch valves are closed. To push fresh, unheated dialysis fluid from pod pump 110 to be heated, positive pressure is applied via air pump 40 and dialysis fluid pinch valve 36a is opened, while all other pinch valves are closed. To push used dialysis fluid from pod pump 110 to drain, positive pressure is applied and drain pinch valve 34 is opened, while all other pinch valves are closed.

For priming, it is contemplated that the cycler provides a prime sensor/bubble detector (not pictured) e.g., an optical or capacitive sensor, which operates with patient line 108 to look for (i) fresh dialysis fluid during priming to know that patient line 108 has been fully primed and (ii) air in patient line 108 during treatment. If air is detected during treatment, the air entrained dialysis fluid may be pulled back along patient line 108 into disposable pumping pod 110, and then pumped out to drain via drain line 104. Alternatively, it is contemplated to allow a certain amount of air to build within pod pump 110, e.g., at the top of the fluid contacting shell, before being delivered to drain.

As discussed above, air pump 40 delivers both positive and negative pressure air to disposable pod pump 110 via a pneumatic arrangement that may include a pneumatic valve manifold. Various pneumatic arrangements 200a-d are illustrated in FIGS. 2A-2D, which are described in more detail below. In particular, as illustrated in FIG. 2A, a positive pressure line 242 leads from positive port 40a (illustrated in FIG. 1A) to a pneumatic valve manifold, while negative pressure line 244 leads from negative port 40b (illustrated in FIG. 1A) to the pneumatic valve manifold. The pneumatic arrangement may include a pneumatic valve manifold, which in the illustrated embodiment includes four pneumatic valves, including pneumatic valves 252a and 252b located between air pump 40 and pod pump 110, and reference chamber valves 254a and 254b located between pod pump 110 and a known volume reference chamber 60 (see FIG. 1A), which is used for fluid volume determinations discussed herein. Pneumatic valves 252a, 252b, 254a and 254b in an embodiment are electrically actuated solenoid valves that open upon being energized so as to operate in a failsafe way.

The pneumatic valve manifold or pneumatic arrangement may also include pneumatic lines 256a, 256b and 256c (hereinafter referred to generally or collectively as pneumatic line(s) 256) linking positive and negative pneumatic valves 252a and 252b and reference chamber valves 254a and 254b. For example, pneumatic line 256a places air pump 40 in fluid communication with the other components of pneumatic arrangement 200a. Pneumatic line 256b places reference chamber 60 in fluid communication with the other components of pneumatic arrangement 200a, and pneumatic line 256c places the pod pump 110 in fluid communication with the other components of pneumatic arrangement 200a. In an embodiment, one of reference chamber valves 254a and 254b pneumatically connects reference chamber 60 to pod pump 110, or more specifically the fluid contacting rigid plastic shell, when needed, while the other of reference chamber valves 254a and 254b pneumatically connects reference chamber 60 to ambient for venting the reference chamber when needed.

In the illustrated embodiment of FIG. 2A, a first pneumatic pressure sensor 248a is located between the pneumatic valve manifold or pneumatic arrangement and disposable pumping pod 110 (pod pressure sensor). A second pneumatic pressure sensor 248b is located between the pneumatic valve manifold or pneumatic arrangement and reference chamber 60 (reference pressure sensor). The pod and reference pressure sensors 248a and 248b are used for the volume determinations discussed herein. Pod pressure sensor 248a is also used to control the pumping pressure of pod pump 110 and to determine an end of stroke for the drawing and discharging of fresh and used dialysis fluid into and from the pod pump. Pressure sensor 248a is located along pneumatic line 256c. Pod pressure sensor 248a enables system 105 to not provide a pressure sensor located along patient line 108, however, such a patient line pressure sensor may be provided if desired, e.g., for redundancy. Reference pressure sensor 248b is located along a pneumatic line 256b, thereby pneumatically linking pneumatic valve manifold or pneumatic arrangement to reference chamber 60.

It should be appreciated that features and components described with respect to FIG. 2A may share the same functions and features, unless specified otherwise. For example, descriptions related to pod pressure sensor 248a as discussed in relation to FIG. 2A may apply to the arrangements 200b-d. Similarly, other components, such as pneumatic lines, valves, etc. may operate and function the same or similar to those as described in relation to a different Figure or example embodiment.

Similar to the example illustrated in FIG. 2A, FIG. 2B illustrates another example pneumatic arrangement with a single reference chamber (e.g., reference chamber 60 of FIG. 1A), which advantageously provides a compact design, which is more compact than the arrangements illustrated in FIGS. 2C and 2D, without requiring additional pressure sensors. Conversely, using a single reference chamber (e.g., reference chamber 60) may require more complex pneumatic switching as the reference chamber is used for both a fluid management system (“FMS”) and as a tank (e.g., storage tank).

As illustrated in FIG. 2B, the pneumatic arrangement in the illustrated embodiment includes five pneumatic valves, including pneumatic valves 252a and 252b located between air pump 40 and pod pump 110, reference chamber valves 254a and 254b located between pod pump 110 and a known volume reference chamber 60 (see FIG. 1A), and pneumatic valve 258 that is positioned along pneumatic bridge line 259. Pneumatic valves 252a, 252b, 254a, 254b and 258 in an embodiment are electrically actuated solenoid valves that open upon being energized so as to operate in a failsafe way.

Similar to the example illustrated in FIG. 2A, pneumatic line 256a places air pump 40 in fluid communication with the other components of pneumatic arrangement 200b. Pneumatic line 256b places reference chamber 60 in fluid communication with the other components of pneumatic arrangement 200b, and pneumatic line 256c places the pod pump 110 in fluid communication with the other components of pneumatic arrangement 200b. In addition to the pneumatic lines 256 linking positive and negative pneumatic valves 252a and 252b and reference chamber valves 254a and 254b, the pneumatic arrangement also includes pneumatic bridge line 259, which bridges pneumatic line 256b in fluid communication with the reference chamber 60 to pneumatic valves 252a and 252b in fluid communication with air pump 40. As illustrated in FIG. 2B, pneumatic arrangement 200b includes pneumatic valve 258 with vent 261 positioned along bridge line 259.

FIG. 2C illustrates an alternative embodiment with two reference chambers. In the illustrated example, one reference chamber (e.g., reference chamber 60 of FIG. 1A) may be used as a tank while the other (e.g., reference chamber 61) is used as a reference. The reference chambers (e.g., reference chamber 60, 61) may alternate between being used as a tank (e.g., storage tank) and used as a reference. By using two reference chambers, the system advantageously remains moderately compact and does not require additional pressure sensors. Additionally, utilizing two reference chambers allows for simple pneumatic switching as one reference chamber may be used for FMS while the other is used as a tank (e.g., storage tank).

Compared to the example illustrated in FIG. 2B, the example pneumatic arrangement 200c with two reference chambers (e.g., reference chamber 61 and reference chamber 60 from FIG. 1A) requires an additional reference chamber 61 and thus has a larger footprint than the previously described examples illustrated in FIGS. 2A-B and additionally requires an extra valve (e.g., pneumatic valve 254c) for routing pressurized air to and from the second reference chamber 61.

As illustrated in FIG. 2C, the pneumatic arrangement in the illustrated embodiment includes six pneumatic valves. The arrangement is similar to that described in relation to FIG. 2B with the addition of pneumatic valve 254c, reference chamber 61 and pressure sensor 248c. Similar to the example in FIG. 2B, pneumatic line 256a places air pump 40 in fluid communication with the other components of pneumatic arrangement 200c. Pneumatic line 256b places reference chamber(s) 60, 61 in fluid communication with the other components of pneumatic arrangement 200c, and pneumatic line 256c places the pod pump 110 in fluid communication with the other components of pneumatic arrangement 200c. Similar to the example in FIG. 2B, pneumatic arrangement 200c also includes pneumatic bridge line 259, which bridges pneumatic line 256b in fluid communication with the reference chambers 60, 61 to pneumatic valves 252a and 252b in fluid communication with air pump 40. Pneumatic valve 254c is a reference chamber valve that pneumatically connects reference chamber 60 to reference chamber 61, which are ultimately pneumatically connected to pod pump 110 via valve 254a (e.g., valve_V3).

FIG. 2D illustrates an alternative embodiment with dedicated pressure tanks. In the illustrated example, two pressure tanks 272, 274 with positive pressure tank 274 dedicated to storing and supplying positive pressure (e.g., pressurized air) and negative pressure tank 272 dedicated to storing and supplying negative pressure (e.g., vacuum or negatively pressurized air). By utilizing dedicated pressure tanks 272, 274, pneumatic arrangement 200d advantageously allows for simple pneumatic switching sequences. Furthermore, example illustrated in FIG. 2D provides a simple control system where the pump 40 is used to top-off the dedicated pressure tanks 272, 274.

Compared to the example illustrated in FIG. 2B, adding dedicated tanks (e.g., positive pressure tank 274 and negative pressure tank 272) to the system increases the footprint of the system and also requires two additional pressure sensors 249a and 249b (e.g., one pressure sensor for each tank).

As illustrated in FIG. 2D, the pneumatic arrangement 200d includes two dedicated pressure tanks (e.g., negative pressure tank 272 and positive pressure tank 274) and five pneumatic valves. Pneumatic valves 252a, 252b, 254a and 254b are in a similar arrangement as described in the previous examples. Pneumatic valve 270 connects the pneumatic lines 257a and 257b corresponding to pressure tanks 272, 274 to the remaining downstream components in the pneumatic arrangement 200d, such as pneumatic line 256b in fluid communication with reference chamber 60 and pneumatic line 256c in fluid communication with pod pump 110. Additionally, each pneumatic arrangement 200a-d illustrates an optional flow sensor 299, which may be used to directly measure instantaneous flow rate of air supplied to or drawn from pod pump 110.

Referring back to the example illustrated in FIG. 2A, the example provides a simple, compact pneumatic system (e.g., there are no pneumatic tanks) with a compact manifold comprising four valves (e.g., valves 252a, 252b, 254a and 254b). However, in the example illustrated in FIG. 2A, pressure transitions are performed using the pump 40, which may be time consuming, and a larger, more powerful pump may be required to provide quicker pressure transitions. However, a larger, more powerful pump adds to the cost and footprint of the pneumatic system, making the system less compact. In an example scenario, the example illustrated in FIG. 2A may perform pressure transitions in approximately 1.5 seconds, whereas having dedicated pneumatic tanks may reduce pressure transition times to approximately 0.1 seconds.

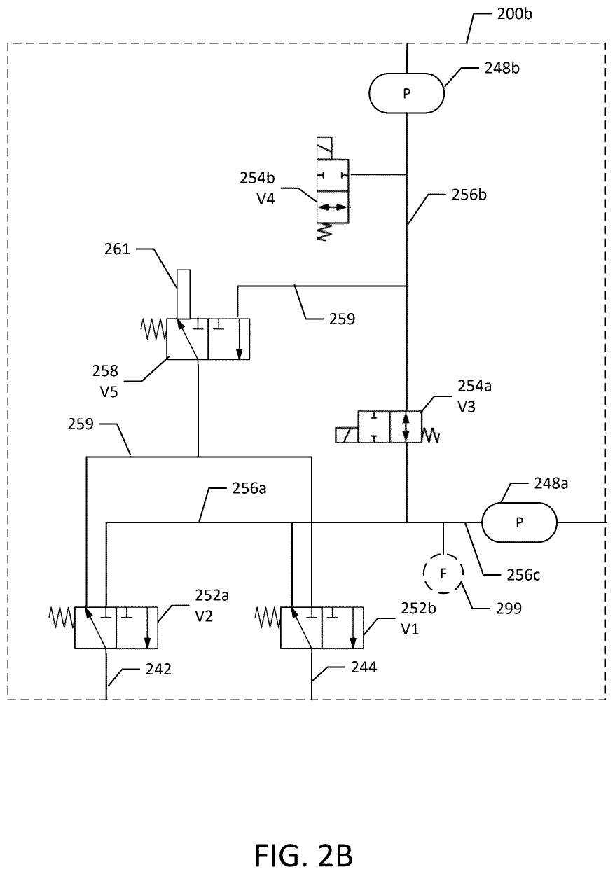

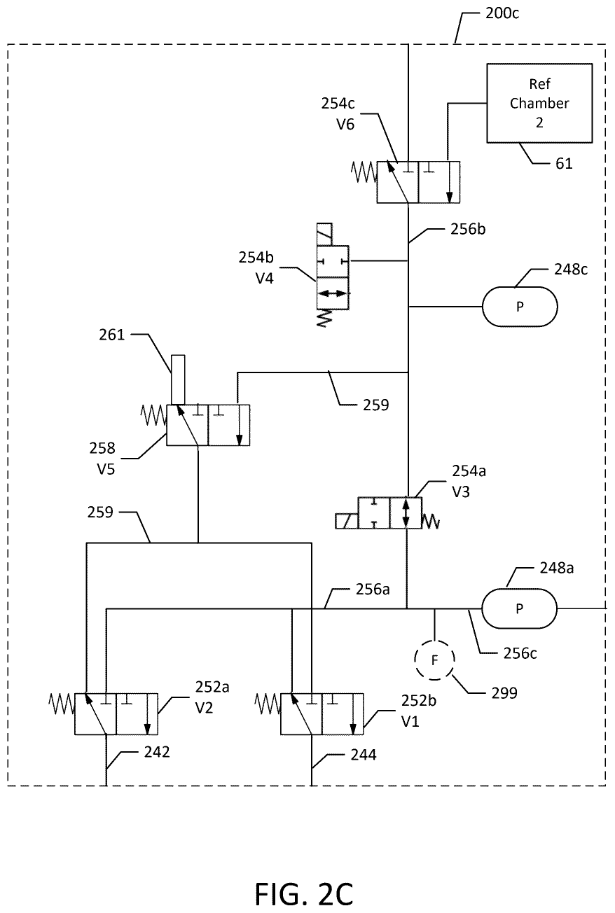

The example illustrated in FIG. 2B employs an additional valve (e.g., valve 258), which allows reference chamber 60 to be used as a pneumatic tank (when otherwise not in use) to reduce pressure transition times such that the transition time of a tankless system (e.g., no pneumatic tanks) is on par with or similar to the 0.1 seconds for systems having dedicated pneumatic tanks. The technique, method and/or process of achieving the improved and reduced pressure transition times are described below in method 300 and illustrated in FIGS. 3, 4A and 4B.

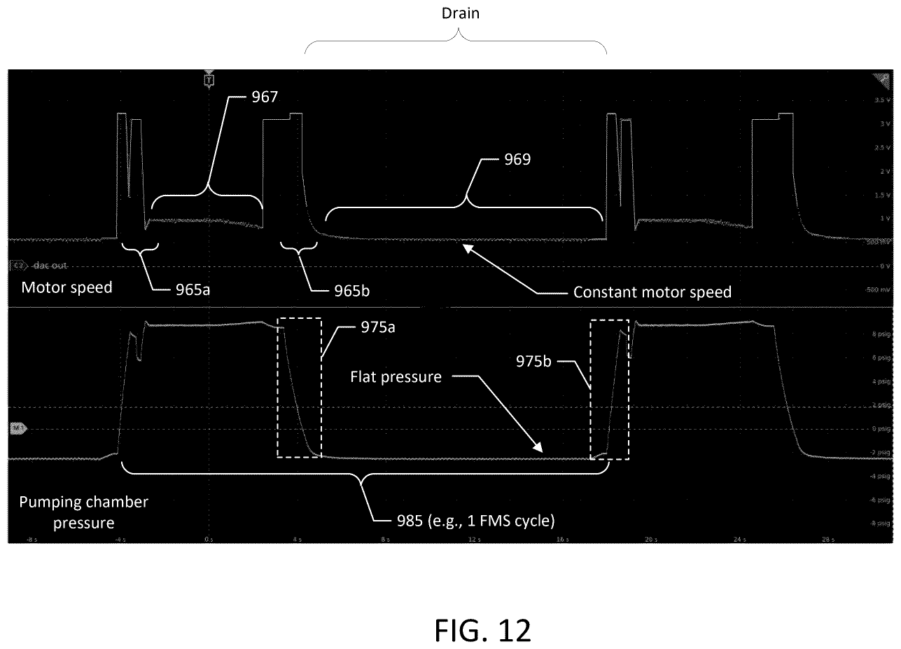

Additionally, the plot of motor speed and pumping chamber pressure illustrated in FIG. 12 further illustrate the advantages of the techniques described in method 300 below. For example, FIG. 12 shows motor speed during periods 965a and 965b of reference chamber usage, motor speed during period 967 while building negative pressure in reference chamber 60, and motor speed during period 969 when building positive pressure in reference chamber 60. FIG. 12 also illustrates the pumping chamber pressure over one FMS cycle 985 and pressure transitions 975a and 975b.

FIG. 3 illustrates a method 300 of determining fresh or used dialysis fluid volume delivery. While discussing the method of FIG. 3, reference is also made to FIGS. 4A and 4B, which are representations of the valve arrangement of FIG. 2B. Control unit 80 is programmed to employ the method 300 for determining how much fresh or used dialysis fluid has been delivered from pod pump 110. In method 300, control unit 80 operates the pump 40 and controls valves (e.gl, valves 252, 254 and/or 258) to create various pressures (e.g., negative pressure and/or positive pressure) in the pump chamber and reference chamber, place the pump chamber and reference chamber in fluid communication to combine and transition pressure, vent the reference chamber and/or pumping chamber, and perform volume calculations on the reference chamber and/or pumping chamber.

At oval 302, method 300 begins. At block 310, control unit 80 creates negative pressure on pump chamber of pod pump 110 and positive pressure on reference chamber 60. Referring back to FIG. 2B, and as further illustrated in FIG. 4A, in an example using reference chamber 60 as a dedicated pressure storage tank, block 310 includes connecting positive port 40a or the “pressure” side of air pump 40 through valves 252a (e.g., valve_V2) and 254a (e.g., valve_V3). Reference chamber 60 is connected to positive port 40a of air pump 40, as indicated by air path 305 (illustrated as a dotted-line and the arrow indicates the direction of flow). Then, valves 252a (e.g., valve_V2) and 252b (e.g., valve_V3) are configured to create suction and air pump 40 runs to create positive pressure on reference chamber 60 and negative pressure on the pumping chamber of pod pump 110, as indicated by air path 315 (illustrated as a dotted-line and the arrow indicates the direction of flow). Once the reference chamber 60 reaches a predetermined pressure (e.g., +14 psig), a vent on valve 258 (e.g., valve_V5) is opened to vent the “pressure” side of air pump 40 via the positive port 40a and valve 252a, which is indicated by air path 325 (illustrated as a solid-line and the arrow indicates direction of flow).

At block 320, control unit 80 maintains pump chamber of pod pump 110 at a first pressure (e.g., −3 psig) and reference chamber 60 at a second pressure (e.g., +14 psig). Referring to FIG. 4A, block 320 includes maintaining the pumping chamber of pod pump 110 at a filling pressure (e.g., −3 psig) until the pod pump 110 is filled with fluid.

At block 330, control unit 80 operates valves to combine reference chamber 60 and the pumping chamber of pod pump 110 to transition pressure. The pressure transition, e.g., pressure transition 975b illustrated in FIG. 12, shows the pumping chamber pressure quickly ramp up from a lower pressure (e.g., −3 psig) to a higher settling pressure (e.g., +5.5 psig or +7 psig) after combining the reference chamber 60 and the pumping chamber of pod pump 110. Referring to FIG. 4A, control unit 80 opens valve 254a (e.g., valve_V3) and closes the remaining valves to place the reference chamber 60 in communication with the pumping chamber of pod pump 110 until the pressure of both chambers settles at a settling pressure (e.g., +7 psig in the case the reference chamber is larger than pod pump 110, whereas for similarly sized chambers the pressure would settle around +5.5 psig). The combination is indicated by air path 335 (illustrated as a solid-line and the arrow indicates the direction of flow).

At block 340, control unit 80 controls valves to vent reference chamber 60 and performs a volume calculation. Referring to FIG. 4A, at block 340, control unit 80 closes valve 254a (e.g., valve_V3) and the reference chamber 60 is disconnected from the pumping chamber of pod pump 110, then reference chamber 60 is vented using valve 254b (e.g., valve_V4). Once the reference chamber 60 is vented, control unit 80 closes valve 254b (e.g., valve_V4) is and opens valve 254a (e.g., valve_V3), and an “initial volume” of the pumping chamber is calculated. For example, control unit 80 may perform the initial volume calculation.

At block 350, control unit 80 controls valves to maintain pressure in the pumping chamber of pod pump 110 at a third pressure (e.g., +9 psig) and to build negative pressure in reference chamber 60. Building negative pressure in reference chamber 60 corresponds to period 967 in FIG. 12, after the usage of reference chamber 965a. Referring to FIG. 4B, positive port 40a of air pump 40 is connected to the pumping chamber of pod pump 110 via valve 252a (e.g., valve_V2), which is indicated by air path 345 (illustrated as a dotted-line and the arrow indicates the direction of flow). Additionally, negative port 40b of air pump 40 is connected to reference chamber 60 via valves 254a (e.g., valve_V3) and valve 252b (e.g., valve_V1), which are controlled via control unit 80. The connection between negative port 40b and reference chamber 60 is indicated by air path 355 (illustrated as a dotted-line and the arrow indicates the direction of flow). While maintaining positive pressure on the pumping chamber of pod pump 110, negative pressure is developed on reference chamber 60 until the reference chamber reaches a reference pressure (e.g., −12 psig).

Once the pressure at reference chamber 60 reaches-12 psig, the “suction” side of the pump 40 is connected to vent 261 using valve 258 (e.g., valve_V5) via control unit 80. Throughout block 350, pressure (e.g., +9 psig) is maintained on the pumping chamber of pod pump 110. Then, once all the fluid is emptied, the pressure in the pumping chamber may be changed to negative, which is described in more detail below at block 360.

At block 360, control unit 80 controls valves to connect reference chamber 60 to the pumping chamber of pod pump 110 to change the pressure in the pumping chamber. The pressure transition, e.g., pressure transition 975a illustrated in FIG. 12, shows the pumping chamber pressure quickly ramp down from a higher pressure (e.g., +9 psig) to a lower settling pressure (e.g., −3 psig) after combining the reference chamber 60 and the pumping chamber of pod pump 110. Referring to FIG. 4B, both the “pressure” side of the pump and the “suction” side of the pump are vented using valves 252a (e.g., valve_V2) and 252b (e.g., valve_V1). After venting, reference chamber 60 (currently at −12 psig) is connected to the pumping chamber (currently at +9 psig) of pod pump 110 via valve 254a (e.g., valve_V3), which is indicated by air path 365 (illustrated as a solid-line and the arrow indicates the direction of flow). When recombined, the pressure between the reference chamber 60 and pumping chamber will settle at approximately −3 psig.

At block 370, control unit 80 performs a volume calculation on the pumping chamber of pod pump 110. Referring to FIG. 4B, reference chamber 60 and the pumping chamber are disconnected using valve 254a (e.g., valve_V3). Then, reference chamber 60 is vented and valve 254b (e.g., valve_V4) is closed. After valve 254b is closed, reference chamber 60 and the pumping chamber are combined using valve 254a (e.g., valve_V3). Once combined, a volume calculation on the pumping chamber is performed. The calculated volume is subtracted from the volume calculated at block 340 to provide the volume displaced.

The blocks may be repeated starting back at block 310 to continue the process, such as connecting the “pressure” side of pump 40 to reference chamber 60, configuring valves 252b (e.g., valve_V1) and 252a (e.g., valve_V2) to create suction, creating a positive pressure on reference chamber 60 and a negative pressure on pumping chamber, etc. Otherwise, the process ends at oval 372. Building positive pressure in reference chamber 60 corresponds to period 969 in FIG. 12, after the usage of reference chamber 965b.

The volume calculations from blocks 340 and 370 may be performed by control unit 80 according to the process described below. For example, control unit 80 may be programmed to employ method 300 for determining how much fresh or used dialysis fluid has been delivered from pod pump 110. In method 300, control unit 80 may measure an initial volume of fresh or used dialysis fluid drawn between flexible sheet 112 and the fluid contacting rigid plastic shell 114 using two sets of pressure measurements and an equation based on the ideal gas law. For example, control unit 80 may take a first set of pressure measurements of (i) the air side of the disposable pumping pod 110 using pod pressure sensor 248a and (ii) reference chamber 60 using reference pressure sensor 248b.

Control unit 80 may take a second set of pressure measurements, where both pod pressure sensor 248a and reference pressure sensor 248b measure the pressure of the combined pneumatic cavity. The difference between the first set of measurements and the second set of measurements is that the first pressure readings are taken before pneumatically connecting pumping pod 110 and reference chamber 60, while the second pressure readings are taken after pneumatically connecting pumping pod 110 and reference chamber 60.

In an example, the volume calculation at block 340, may be determined according to the following equation. With all values on the right side of the following equation (based on the ideal gas law) known or measured, including the known volume of reference chamber 60, control unit 80 may calculate the volume of fresh or used dialysis fluid pulled into disposable pod pump 110 is as follows:

$${V}_{fluid initial} = {V}_{reference chamber} * ( {P}_{ref final} - {P}_{ref initial} ) / ( {P}_{pump initial} - {P}_{pump final} ) \qquad ( Eq . 1 )$$

Additionally, control unit 80 may be programmed to measure a final volume of fresh or used dialysis fluid located between flexible sheet 112 and fluid contacting rigid plastic shell 114 using the same two sets of pressure measurements and an equation based on the ideal gas law. For example, in a first set of pressure measurements, control unit 80 takes pressure measurements of (i) the air side of the disposable pod pump 110 using the pod pressure sensor 248a and (ii) reference chamber 60 using reference pressure sensor 248b.

In a second set of pressure measurements, both pod pressure sensor 248a and reference pressure sensor 248b measure the pressure of the combined pneumatic cavity. The difference between the first set of measurements and the second set of measurements is that the first pressure readings are taken before pneumatically connecting pod pump 110 and reference chamber 60, while the second pressure readings are taken after pneumatically connecting pod pump 110 and reference chamber 60.

With all values on the right side of the following equation (based on the ideal gas law) known or measured, including the known volume of reference chamber 60, control unit 80 calculates the volume of fresh or used dialysis fluid remaining in pod pump 110 after the discharge stroke as follows:

$${V}_{fluid final} = {V}_{reference chamber} * ( {P}_{ref final} - {P}_{ref initial} ) / ( {P}_{pump initial} - {P}_{pump final} ) \qquad ( Eq . 2 )$$

In an example, control unit 80 calculates the volume of fresh or used dialysis fluid pumped from pod pump 110 by calculating the difference between the calculated fluid volume after drawing in fluid (V_{fluid initial}) and the calculated fluid volume after pumping fluid out (V_{fluid final}) pumping.

The systems and methods described above with reference to FIGS. 1A, 1B, 2A, 2B, 3, 4A and 4B advantageously provide improved flow rate of single chamber pneumatic APD systems. One limitation of a typical pod pump fluid management system (“FMS”) is that the pump is used to make pressure transitions, and each pressure transition may last for a few seconds (typically 1.5-2 seconds). By implementing the systems and methods described herein, the limitation of previous systems and techniques may be overcome and flow rates may advantageously be improved by up to 15%, thereby reducing therapy time. In the specific examples described above, the pod pump FMS is a simple pneumatic system that functions without additional tanks or reservoirs, making the system compact and cost-effective. Conversely, other approaches may use dedicated tanks to achieve the same advantages and results, but dedicated tanks disadvantageously make the system bulkier and require additional valves.

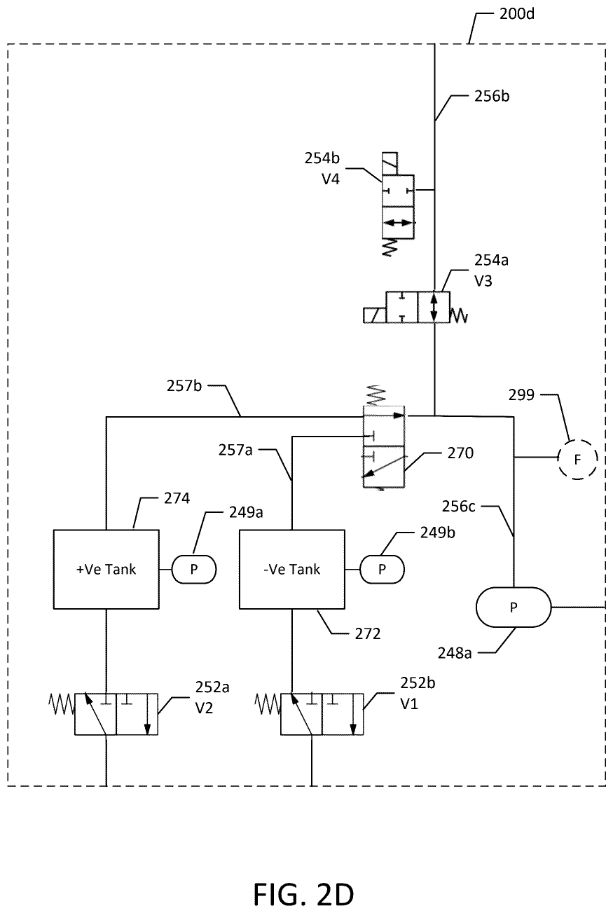

FIG. 5 illustrates a drain logic, process flow and/or method 500 of detecting an occlusion (e.g., a pneumatic or fluid line kink) or an end of drain condition (referred to herein generally as “end of drain”) based on motor speed. For example, an occlusion or “end of drain” may be determined based on motor speed and flow rate of air to compute a flow rate of dialysate fluid. Under constant pressure, air flow rate is effectively the same as the fluid flow rate, such that when pressure is maintained, instantaneous flow rate of the pump is effectively the same as flow rate of a fill or drain operation. Typically, during normal operating situations when there is sufficient fluid in peritoneum, motor speed is constant.

The method 500 described below provides an advantage(s) over existing techniques that use flow rate to detect (i) an occlusion, such as a king in a fluid line or tube and/or (ii) an “end of drain.” Instead of using flow rate, which is often more time consuming thereby increasing therapy time and increasing the likelihood of drain pain, the technique described by method 500 below advantageously provides a quick detection of (i) an occlusion, such as a king in a fluid line or tube and/or (ii) an “end of drain” and also reduces drain time and thus therapy time.

Thus, referring back to FIGS. 2A-2D, a flow sensor (e.g., flow sensor 299) may be used to directly measure instantaneous flow rate. As noted above, under constant pressure air flow rate a flow sensor may be used to accurately monitor flow rate or pump speed, and thus may also be used to measure or approximate flow rate. More specifically, when pressure is constant, the volume of air pushed into the chamber is equal to volume of air pumped (at the same pressure). Based on this relationship, the flow rate of air into the pumping chamber is equal to instantaneous drain flow rate. The flow rate of the pump is also a function of pump speed and can be determined based on the relationship between flow rate and pump speed.

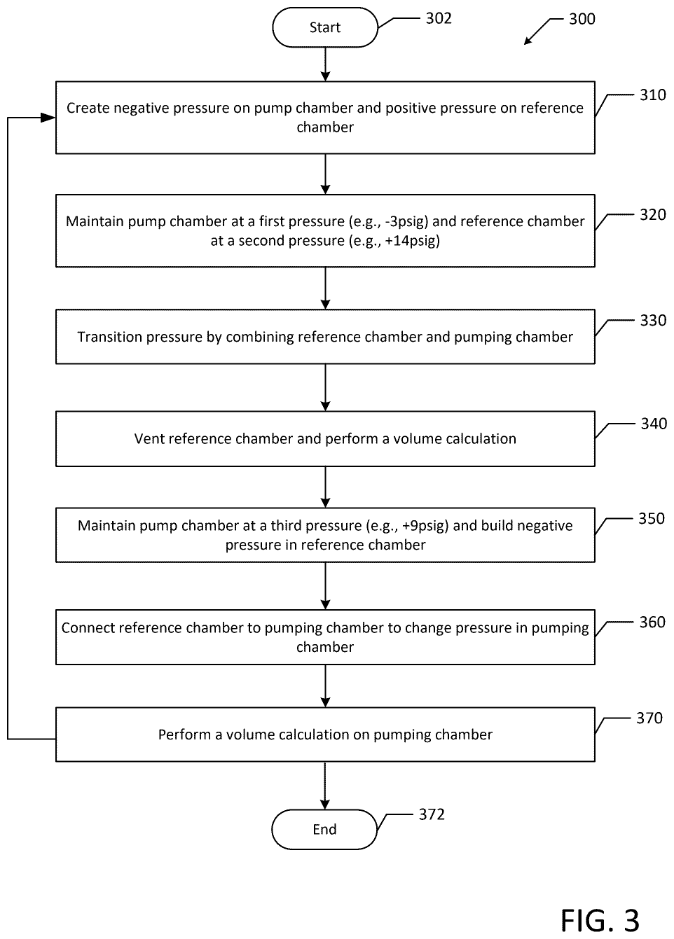

Converse to detecting an occlusion or “end of drain” using flow rates, which is an effective method, but often is more time consuming and entails continuing to apply suction to a patient's peritoneum until the average flow rate is detected low enough to declare that a drain cycle is complete or “drain is complete.” The additional time consumed by detecting an occlusion or “end of drain” using flow rates translates into additional therapy time for the patient and may lead to drain pain or discomfort experienced by the patient. In some instances, a patient may be exposed to an additional 30-40 seconds of unnecessary suction from a conventional system that relies on rolling averages of flow rate and/or transitions from high-flow to low-flow and then finally to no-flow. For example, each transition (e.g., high-flow to low-flow) may take approximately 30 seconds and in some cases even longer. By using motor speed, occlusions and “end of drain” can be determined more quickly than with flow rates, thereby reducing detection time and therapy time and thereby reducing discomfort (e.g., drain pain) to the patient. Specifically, the method illustrated in FIG. 5 advantageously provides simple and quick detection of an occlusion (e.g., kink) and/or quick, near immediate determination or calculation of the instantaneous flow rate and “end of drain”, reduces drain time and therapy time, reduces drain pain when “smart pressure” controls are implemented (see FIG. 6), and provides a simple process for measuring instantaneous flow rate.

Drain pain may include pain or discomfort caused by suction that induces pain toward the end of a drain cycle. For example, a tip of an intraperitoneal portion of a catheter may lie against the patient's parietal peritoneum towards the end of a drain cycle, which may induce drain pain. In other cases, the application of negative pressure and/or suction to the patient's parietal peritoneum (which may be very sensitive) may induce drain pain, especially towards the end of each drain cycle.

FIG. 5 illustrates an example drain logic method 500, which transitions to pressure control (e.g., to reduce drain pain) when pump speed is zero. Drain logic method 500 starts at oval 502. At block 504, fluid is loaded into the pod pump. For example, referring to FIGS. 1A and 1B, fluid is loaded or drawn into the pod pump 110 through control of air pump 40 and fluid pinch valves 36a-c.

To draw fresh dialysis fluid into pod pump 110, negative pressure is applied via air pump 40 and one of dialysis fluid pinch valves 36a to 36c is opened, while all other pinch valves are closed. To draw fresh, heated dialysis fluid into pod pump 110, negative pressure is applied and dialysis fluid/heater pinch valve 36a is opened, while all other pinch valves are closed. To draw used dialysis fluid from patient P into pod pump 110, negative pressure is applied and patient valve 38 is opened, while all other pinch valves are closed. To push fresh, heated dialysis fluid from pod pump 110 to patient P, positive pressure is applied and patient valve 38 is opened, while all other pinch valves are closed. To push fresh, unheated dialysis fluid from pod pump 110 to be heated, positive pressure is applied via air pump 40 and dialysis fluid pinch valve 36a is opened, while all other pinch valves are closed. To push used dialysis fluid from pod pump 110 to drain, positive pressure is applied and drain pinch valve 34 is opened, while all other pinch valves are closed.

Then, at block 506, fluid is pumped at a constant pressure using closed loop control of the motor. For example, fluid may be pumped or pushed from pod pump 110 through control of air pump 40 and fluid pinch valves as described above with reference to FIGS. 1A and 1B. While fluid is pumped at constant pressure, speed of the pump is monitored at block 508. In an example, air pump 40 may be electrically actuated such that the amount of current delivered to the air pump determines the speed of the pump and the pressure and flowrate of air delivered to pod pump 110. The speed of air pump 40 is measureable, e.g., via monitoring a moving part of the air pump such as a piston, membrane or shaft rotation speed, and wherein the measured speed is proportional to dialysis fluid filling flowrate. Alternatively or additionally, the speed of air pump 40 may be known or assumed from a speed commanded by control unit 80. In an example, the control unit 80 (see FIGS. 1A, 1B) may be configured to sensor and/or monitor the speed of the pump.

At block 510, the drain logic method 500 includes determining whether the pump speed is zero. Determining whether the pump speed is zero may be performed according to any of the examples provided above with respect to block 508. If the pump speed is zero, the drain logic lowers the pump pressure to reduce drain pain at block 512. When the pump speed is zero, the pump pressure is already negative, since negative pressure is used to pull used dialysis fluid from the patient P. If the pump speed is still greater than zero, the drain logic method 500 determines if the pump speed is lower than a predetermined threshold at block 514. In an example, the predetermined threshold may be 0.2V as the pump speed is controlled through voltage. In an example, the threshold may range from greater than OV up to 0.2V.

If the pump speed is equal to or greater than the predetermined threshold, the drain logic determines whether a predetermined pumping time has elapsed at block 518. The pumping time may be programmed and designated based on the type of treatment selected, patient parameters, etc. In an example, pumping time may be 30 seconds. In other examples, the pumping time may be more or less than 30 seconds (e.g., anywhere from 10 seconds to 90 seconds). If pumping time has not elapsed at block 518, then the drain logic method 500 reverts back to the determination at block 510 to determine if the pump speed is now zero and continues to cycle through this loop until either the pump speed is less than the threshold speed or the pumping time has elapsed. If the pumping time has elapsed at block 518, then the drain logic method 500 starts back at block 504, which includes loading fluid into the pod pump.

It should be appreciated that block 510 of method 500 may only be initiated at the end of a drain cycle (e.g., low flow rate). If a low flow rate does not exist and there is no indication that the patient is nearing end of drain, the method 500 will not advance to block 510. For example, between blocks 508 and 510, the method 500 may include a determination that the flow rate is below a predetermined threshold indicating that an “end of drain” condition may be present.

Referring back to block 510, if the pump speed is already zero, then the drain logic includes lowering pressure to reduce drain pain at block 512. For example, pressure may be reduced to 0.5 psig. Furthermore, referring back to block 514, if the pump speed is less than the predetermined threshold, the drain logic may wait until the pump speed is zero at block 516. After either of blocks 512 and 514, drain logic or method 500 continues at block 520 with a determination whether a predetermined time interval (e.g., 10 seconds in the illustrated example) has elapsed with the pump speed at zero. The drain logic or method 500 continues to wait for the predetermined time interval to elapse with the pump speed at zero before making a determination that a “push back” procedure was successful at block 522.

For example, “push back” procedure involves a scenario when the pump is no longer experiencing flow, which may be due to (i) an occlusion (e.g., kinked line) or (ii) the patient's peritoneum being empty (e.g., no fluid left to drain from the peritoneum). The pump may attempt to push back a small bolus of fluid to determine whether condition (i) or (ii) is present. Specifically, if a push back procedure is successful, that indicates that condition (ii) the patient's peritoneum being empty is satisfied and the drain cycle is complete at block 524. If the line was occluded or kinked, the pump would be unable to push back the bolus of fluid.

Conversely, if the push back procedure is unsuccessful at block 522, drain logic or method 500 determines that an occlusion condition exits, e.g., detects an occlusion at block 526. If an occlusion is detected, method 500 may include activating an alarm at block 528 to alert the patient and/or practitioner of the detected occlusion. The patient may also be prompted to look for a kink or occlusion in reusable portion of drain line. In another example, the cycler may slow down or stop the present drain flow in the event of an occlusion detected at block 526.

FIG. 6 illustrates an example process or method 600 for closed loop pressure control for reaching and maintaining a target pressure. Referring back to FIGS. 2A-2D, a flow sensor (e.g., flow sensor 299) may be used to directly measure instantaneous flow rate, and under constant pressure the air flow rate is the same or nearly identical as the fluid flow rate. The method 600 starts by passing a target pressure at block 610 to block 620 where error between current pressure and the target pressure is determined. For example, the difference or percent error between the actual pressure and target pressure is determined at block 620. When draining fluid, the target pressure is zero (e.g., going from negative pressure or suction to a zero pressure). Typically, as pressure builds (e.g., becomes less negative), the error trends towards zero. Furthermore, when pressure is maintained, instantaneous flow rate of the pump is the same as the flow rate of fill or drain.

The error at block 620 may be amplified by a gain amplifier or a proportional integral (“PI”) loop gain at block 630. The PI loop gain is a control loop mechanism employing feedback. For example, an error value e (t) may be continuously calculated as the different between a desired setpoint and a measured process variable. In the present example, the target pressure at block 610 is the desired setpoint and the pressure feedback at block 660 is the measured process variable, which may be continuously used to calculate the error at block 620. A correction may be applied based on proportional and integral terms. In an example, the PI loop gain at block 630 may be determined as an output where the output is the sum of proportional gain multiplied by the error and the integral gain multiplied by the error (e.g., output=(proportional gain x error)+ (integral gain x error)). The correction or adjustment determined at block 630 may be applied to adjust pressure until the measured pressure matches or is within an acceptable tolerance of the target pressure.

Then, information regarding motor speed is obtained at block 640. Additionally, motor speed may be adjusted at block 640 according to the feedback from block 660 described in more detail below. Motor speed may be monitored and obtained through any of the techniques described herein (e.g., via control unit 80). In some examples, motor speed may be monitored and adjusted based on voltage (e.g., voltage provided to the motor). During normal conditions, when there is sufficient fluid in the patient's peritoneum, motor speed is constant. As noted above, under constant pressure, the air flow rate matches the fluid flow rate. The flow rate may be monitored via a flow sensor or the pump speed may be used to approximate flow rate (e.g., flow rate of pump is a function of pump speed). Specifically, when pressure is constant, the volume of air pushed into the pumping chamber of pod pump 110 is equal to the volume of air pumped (e.g., from air pump 40) at the same pressure. Therefore, flow rate of air into the pumping chamber of pod pump 110 is equal to the instantaneous drain flow rate.

Information regarding the pneumatic pump may also be obtained at block 650. Additionally, other information regarding the pneumatic pump may be determined at block 650 based on the relationship between the air and fluid flow rates noted above. For example, pneumatic pump information at block 650 may be determined based on the motor speed obtained at block 640.

As illustrated in example method 600, the information obtained regarding motor speed and/or the pneumatic pump (e.g., air pump 40) may provide pressure feedback details at block 660, which can be used to make another error determination at block 620. For example, based on (i) the correction or adjustment determined at block 630, (ii) the relationship between motor speed and flow rate of the pump, and (iii) flow rate of air into the pumping chamber equaling the instantaneous drain flow rate, the pressure may quickly trend to the target pressure of zero when performing method 600. The process illustrated in FIG. 6 repeats until the target pressure is reached and maintained.

The methods described in FIGS. 5 and 6 provide various advantages such as faster “drain complete” or empty detection, reduced therapy times, improved drain logic to reduce drain pain, “smart pressure” control to reduce drain paint by reducing drain pressure as flow rate decreases, simple techniques of measuring instantaneous flow rate, and real time instantaneous flow rate measurement to improve the speed of occlusion detection.

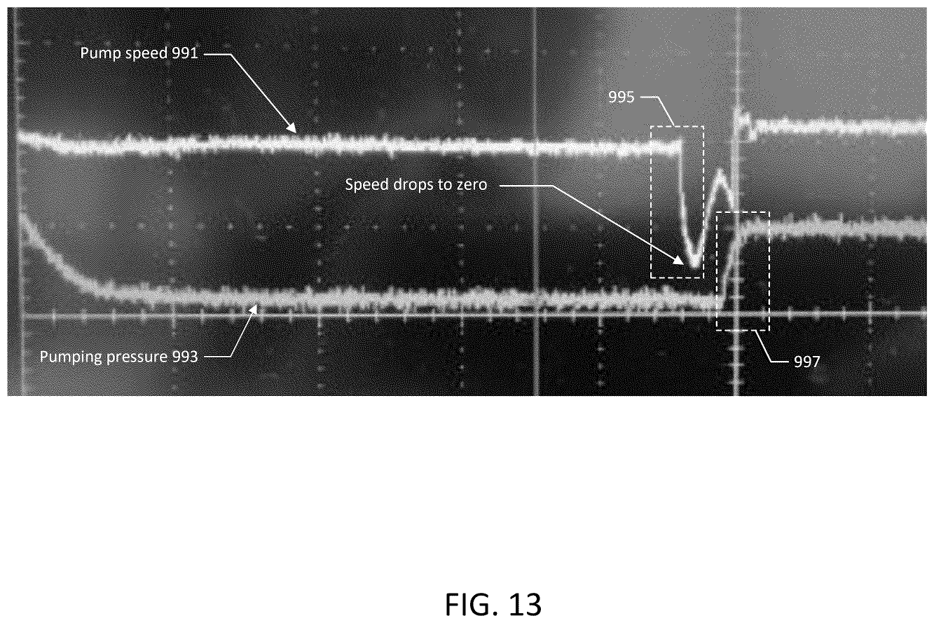

Referring to FIG. 12, a normal drain profile is illustrated which indicates that motor speed is relatively constant when there is sufficient fluid in a patient's peritoneum. As mentioned above, under constant pressure (e.g., flat pressure profile in FIG. 12), air flow rate is essentially equal to the flow rate of the fluid. By using the correlation or direct relationship of flow rate of air into the pumping chamber of pod pump 110 being equal to the instantaneous drain flow rate, the improved drain logic described in method 500 advantageously reduces drain pain compared to previous techniques. FIG. 13 further illustrates the advantages of methods 500 and 600. Specifically, as the pump speed 991 drops to zero, which indicates that the patient's peritoneum is empty or “end of drain” at region 995, the pumping pressure 993 is quickly reduced at region 997 (e.g., becomes less negative), which is depicted by an increase in the pumping pressure. Since the pumping pressure is negative (e.g., suction) to perform the drain, reducing the magnitude of the negative pressure for suction is shown as an increase in pressure. The example test results depicted in FIG. 13 show an increase in voltage, which corresponds to a reduction in negative pressure.

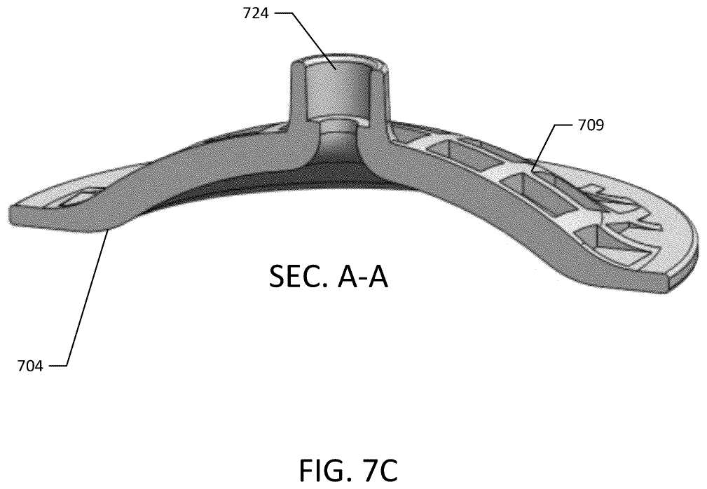

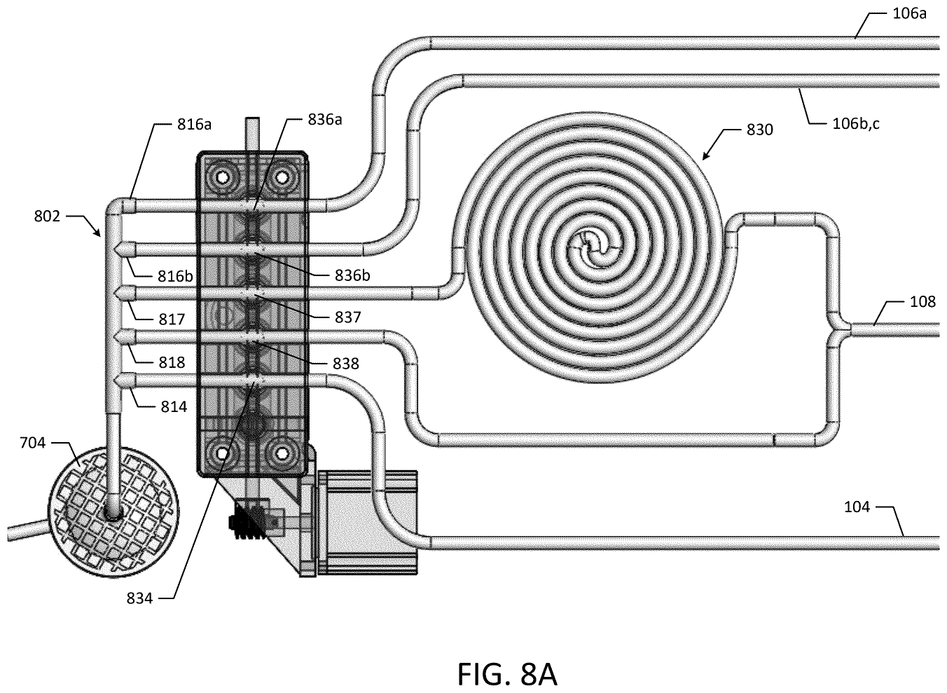

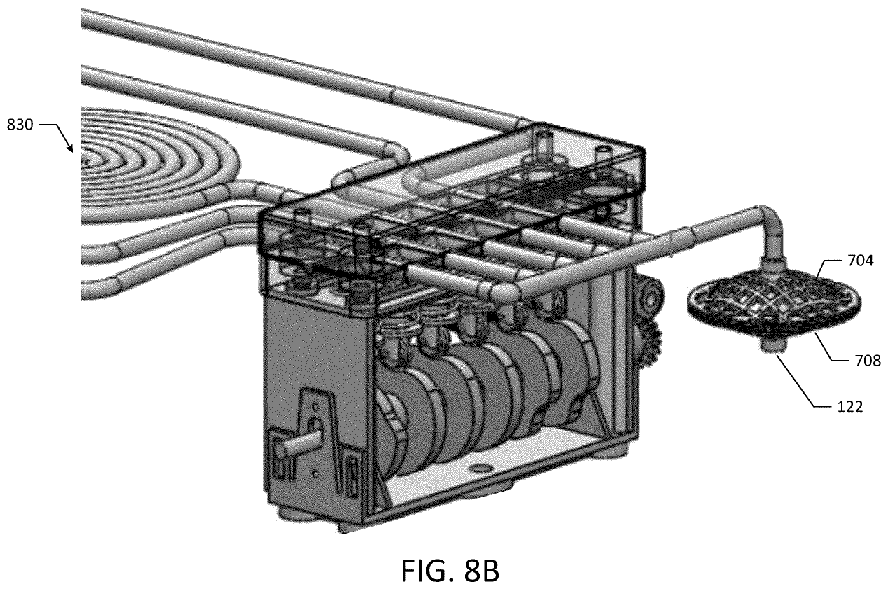

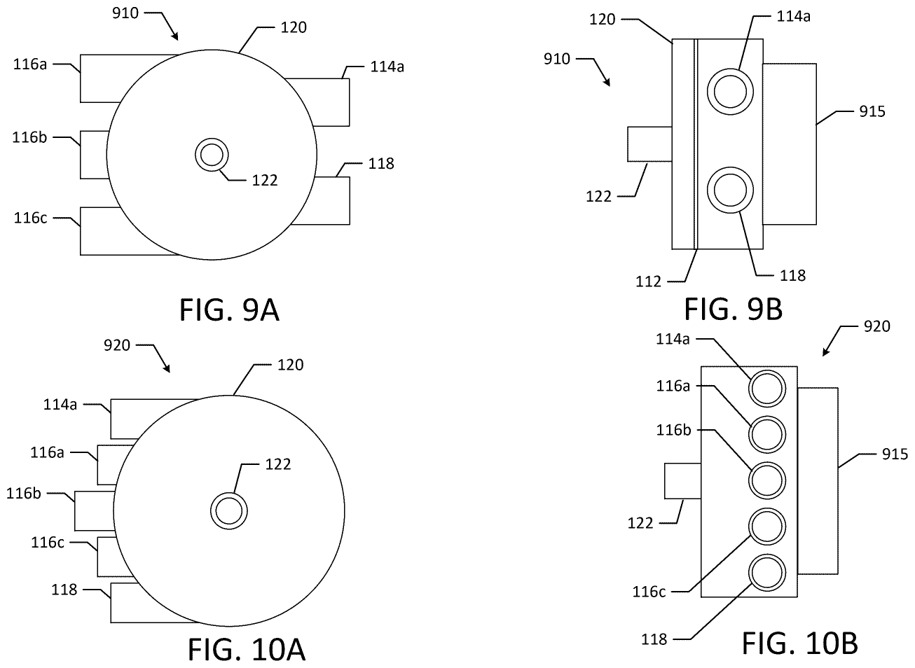

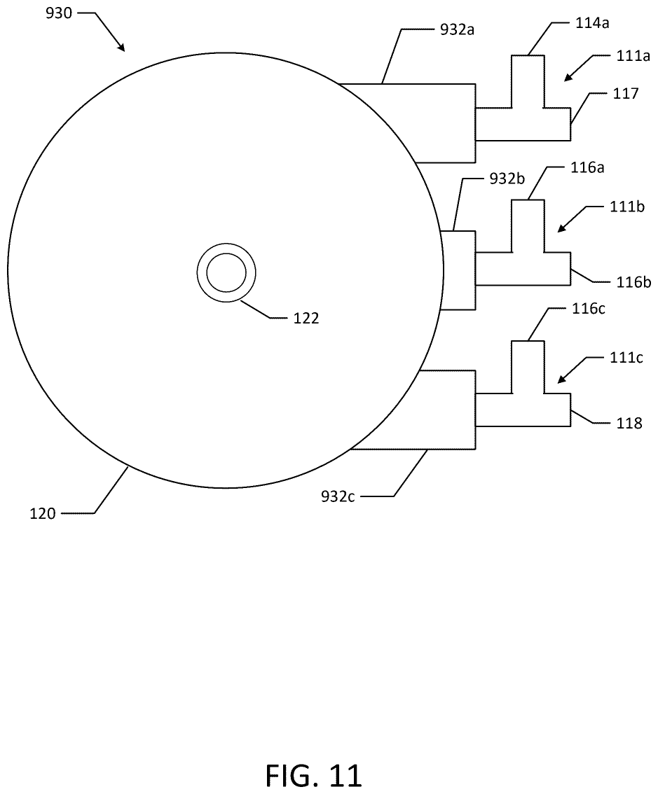

FIGS. 7A-C, FIGS. 8A-B, FIGS. 9A-B, FIGS. 10A-B, and FIG. 11 illustrate that pod pump 110 may be constructed in multiple ways. Specifically, various examples described different designs for an APD disposable for single chamber pneumatic bases systems. Typically, an APD disposable is tasked with pumping fluid, metering fluid, and valving fluid in a controlled manner (e.g., at a controlled pressure). However, it may be challenging to achieve each of the above functions in disposable. The disposable pod pump(s) 110 described below are configured to (i) pump fluid, (ii) meter fluid and (iii) valve fluid at a controlled pressure. Some existing disposables use two small chambers to pump fluid, but the examples provided below use a single, often larger chamber to pump fluid, which simplifies the design and reduces production costs. The examples provided below also advantageously allow for higher flow rates, which may reduce therapy time. The higher flow rates are a result of larger chamber size(s) and higher pressures that are achievable with the examples provided below.

In FIGS. 7A-C, pod pump 710, which may be referred to herein generally as pod pump 110, includes a fluid contacting shell 704 and an additional pneumatic shell 708, which are sealed together to hold flexible sheet, diaphragm or membrane 112 in a sealed manner therebetween on an internal surface of the pneumatic shell 708. The shells 704, 708 may be made from Polyethylene terephthalate glycol (“PETG”) and may include reinforcement ribs 709 that extend outwardly from and span across the outside, external surface of the shells 704, 708. In the illustrated example, reinforcement ribs 709 are arranged in a cross-knit or waffle pattern. Ribs 709 may advantageously provide additional strength and durability to shells 704, 708 while reducing overall material used to construct shells 704, 708, thereby reducing material and production costs. It should be appreciated that other designs, configurations and arrangements of reinforcement ribs 709 or other reinforcement structures may be implemented.

A central port 122 is provided in pneumatic shell 708, which is a pneumatic port that communicates pneumatically with air pump 40 and pneumatic valve manifold or pneumatic arrangement(s) 200a-d, e.g., seals to or within a pneumatic aperture provided in an actuation surface. Here, air and pneumatic pressure resides between pneumatic shell 708 and flexible membrane 112. Fluid contacting shell 704 also includes a central port 724, which may lead to a manifold, as illustrated in FIG. 8A, which may include a drain port 814, dialysis fluid ports 816a to 816c and patient port 818. Drain port 814, dialysis fluid ports 816a to 816c and patient port 818 may have the same features and functions as drain port 114a, dialysis fluid ports 116a-c and patient port 118 described herein. In an embodiment, flexible sheet 112 is attached, e.g., ultrasonically welded, heat sealed or adhered to one of the shells 704 or 708, after which that subassembly is attached, e.g., ultrasonically welded, heat sealed or adhered to the other one of rigid shells 704 or 708. In the embodiment of FIGS. 7A-C, plastic sheet 112 is pulled pneumatically into pneumatic shell 708 to draw fresh or used dialysis fluid into the fluid contacting shell 704. Plastic sheet 112 is again pushed pneumatically into fluid contacting shell 704 to dispel or discharge fresh or used dialysis fluid from pod pump 110.

FIGS. 8A and 8B illustrate manifold 802 downstream of central port 724 of fluid contacting shell 704. The manifold may be a rigid PVC manifold that connects to various fluid lines passing through corresponding pinch valves (e.g., pinch valve 836a, 836b, 837, 838, and 834). Manifold 802 includes drain port 814, dialysis fluid ports 816a to 816c (one of which may be a dialysis fluid heating port, such as port 816b), and patient port 818. In the illustrated example, dialysis fluid port 816a connects to fluid line 106a, which passes through pinch valve 836a, and ultimately leads to container 102a. Dialysis fluid port 816b connects to fluid line 106b, which passes through pinch valve 836b, and ultimately leads to last fill bag (e.g., container 102b or container 102c). Heat port 817 connects to heater line that forms an inline heater 830 (e.g., an inline spiral tube heater), which eventually connects to patient line 108. Patient port 818 connects to patient line 108, which passes through pinch valve 838, and ultimately leads to the patient's peritoneum. The drain port 814 of manifold 802 connects to drain line 104, which passes through pinch valve 834, and eventually leads to a drain.

FIGS. 9A and 9B illustrate a disposable pod pump 910, which may include the same features and functions as disposable pod pump(s) 110 described herein, such as central port fluid contacting shell 114, flexible sheet, diaphragm or membrane 112, pneumatic shell 120, central port 122 and flid ports (e.g., drain port 114a, dialysis fluid ports 116a to 116c and patient port 118). Pod pump 910 may be referred to herein generally as pod pump 110. The fluid contacting shell and the pneumatic shell may be rigid plastic shells that are sealed together to hold flexible sheet, diaphragm or membrane 112 in a sealed manner therebetween.

In the illustrated example, pod pump 910 includes a stopcock handle 915. Stopcock handle 915 may be controlled by a motor (e.g., stepper motor) configured to align and open one of the fluid ports (e.g., drain port 114a, dialysis fluid ports 116a to 116c and patient port 118) while closing all others. In an example, stopcock handle 915 is press-fit inside a housing of pod pump 910.

FIGS. 10A and 10B illustrate a disposable pod pump 920, which is similar to disposable pod pump 910, but instead of the ports being positioned about both sides of the shell(s) or housing, the ports are aligned on one a single side. This configuration is similar to the configuration illustrate in FIGS. 1A and 1B. Pod pump 920 may be referred to herein generally as pod pump 110.

FIG. 11 illustrates another example disposable pod pump 930, similar in construction to pod pumps 910 and 920. Pod pump 930 may be referred to herein generally as pod pump 110. Instead of dedicated fluid ports for each fluid line, pod pump 930 includes three multi-fluid ports 932a-c. Multi-fluid port 932a supports a multiport stopcock valve 111a that includes a drain port 114a and a heater bag port 117. Multi-fluid port 932b supports a multiport stopcock valve 111b that includes a fluid port 116a associated with a first supply bag or fluid container 102a and fluid port 116b associated with a second supply bag or fluid container 102b. Multi-fluid port 932c supports a multiport stopcock valve 111c that includes a fluid port 116c associated with a third supply bag or fluid container 102c and a patient port 118.

Even though the example illustrated in FIG. 11 includes multiple multiport stopcock valves, the example advantageously requires less actuation stepper motors than the examples described in FIGS. 9A, 9B, 10A and 10B.

It should be appreciated that any of the pinch valves described herein (e.g., pinch valve 34, 36a to 36c or 38 described above), may be formed from metal, such as stainless steel, steel and/or aluminum, and/or molded from plastic, such as polyvinylchloride (“PVC”) or a non-PVC material, such as polyethylene (“PE”), polyurethane (“PU”) or polycarbonate (“PC”).

It should be understood that various changes and modifications to the presently preferred embodiments described herein will be apparent to those skilled in the art. For example, pneumatic valves may be used instead of electrically actuated pinch valves. In another example, separate positive and negative air pumps may be used to supply positive and negative air pressure instead of single air pump 40. In a further example, heater 30 could be located remote from cycler 20a. Such changes and modifications can be made without departing from the spirit and scope of the present subject matter and without diminishing its intended advantages. It is therefore intended that such changes and modifications be covered by the appended claims.

The invention is claimed as follows:

1. A peritoneal dialysis system comprising:
a cycler including:
a pneumatic valve manifold,
an air pump positioned and arranged to supply positive and negative pneumatic pressure to the pneumatic valve manifold without intervening pneumatic storage,
at least one pneumatic pressure sensor positioned and arranged to detect pneumatic pressure, and
a control unit configured to use an output of the pneumatic pressure sensor as feedback to adjust the air pump according to a set pneumatic pressure; and
a disposable set including a pod pump having a flexible sheet attached to at least one pump housing, one side of the flexible sheet positioned and arranged during operation to receive pneumatic pressure via the air pump and pneumatic valve manifold.

2. The peritoneal dialysis system of claim 1, wherein the flexible sheet is attached to a single pump housing.

3. The peritoneal dialysis system of claim 2, wherein the single pump housing includes a drain port, at least one dialysis fluid port, and a patient port.

4. The peritoneal dialysis system of claim 1, wherein the at least one pump housing includes a first pump housing and a second pump housing, and wherein the flexible sheet is positioned and arranged between the first pump housing and the second pump housing.

5. The peritoneal dialysis system of claim 4, wherein at least one of the first pump housing and the second pump housing includes a drain port, at least one dialysis fluid port, and a patient port.

6. The peritoneal dialysis system of claim 4, wherein the first pump housing has an internal surface facing the flexible sheet and an external surface, the external surface including a first plurality of reinforcement ribs extending along the external surface in a first direction and a second plurality of reinforcement ribs extending along the external surface in a second direction.

7. The peritoneal dialysis system of claim 6, wherein the second pump housing has an internal surface facing the flexible sheet and an external surface, the external surface including a plurality of reinforcement ribs extending along the external surface.

8. The peritoneal dialysis system of claim 1, wherein the pneumatic valve manifold includes a plurality of valves, the plurality of valves being at least one of pinch valves, stopcock valves, and volcano valves.

9. The peritoneal dialysis system of claim 1, wherein the disposable set includes a plurality of ports in fluid communication with the pod pump, the at least one pump housing includes a first pump housing and a second pump housing, and the second pump housing includes a stopcock handle configured to place a first port of the plurality of ports in an open arrangement while placing each of the other ports of the plurality of ports in a closed arrangement.

10. The peritoneal dialysis system of claim 9, wherein the cycler further includes a stepper motor configured to control a position of the stopcock handle, wherein the position of the stopcock handle determines which port of the plurality of ports is in the open arrangement while each of the other ports of the plurality of ports is in the closed arrangement.

11. The peritoneal dialysis system of claim 9, wherein the plurality of ports includes a drain port, at least one supply port, and a patient port.

12. The peritoneal dialysis system of claim 1, wherein the pod pump includes a housing having a plurality of multi-function combination ports.

13. The peritoneal dialysis system of claim 12, wherein the plurality of multi-function combination ports includes at least one of a first combination port configured to fluidly communicate with a drain port and heater port, a second combination port configured to fluidly communicate with a first supply port and second supply port, and a third combination drain port configured to fluidly communicate with a third supply port and a patient port.

14. The peritoneal dialysis system of claim 12, wherein the plurality of multi-function combination ports includes a first combination port configured to selectively actuate communication between a drain port and at least one of a heater port, a drain port, and a patient port.

15. The peritoneal dialysis system of claim 12, wherein the plurality of multi-function combination ports includes a first combination port configured to selectively actuate communication between a supply port and at least one of a heater port, an additional supply port, a drain port, and a patient port.

16. The peritoneal dialysis system of claim 12, wherein the plurality of multi-function combination ports includes a first combination port configured to selectively actuate communication between a drain port and at least one of a heater port, a supply port, and a patient port.

17. A peritoneal dialysis system comprising:
a cycler including:
a pneumatic valve manifold,
an air pump positioned and arranged to supply positive and negative pneumatic pressure to the pneumatic valve manifold without intervening pneumatic storage,
at least one pneumatic pressure sensor positioned and arranged to detect pneumatic pressure, and
a control unit configured to use an output of the pneumatic pressure sensor as feedback to adjust the air pump according to a set pneumatic pressure; and
a disposable set including a pod pump, a plurality of ports and a stopcock handle configured to selectively open and close one or more ports of the plurality of ports.

18. The peritoneal dialysis system of claim 17, wherein the plurality of ports includes a drain port, at least one dialysis fluid port, and a patient port.

19. A peritoneal dialysis system comprising:
a cycler including:
a pneumatic valve manifold,
an air pump positioned and arranged to supply positive and negative pneumatic pressure to the pneumatic valve manifold without intervening pneumatic storage,
at least one pneumatic pressure sensor positioned and arranged to detect pneumatic pressure, and
a control unit configured to use an output of the pneumatic pressure sensor as feedback to adjust the air pump according to a set pneumatic pressure; and
a disposable set including a pod pump having a flexible sheet and a plurality of multi-function combination ports, one side of the flexible sheet positioned and arranged during operation to receive pneumatic pressure via the air pump and pneumatic valve manifold.

20. The peritoneal dialysis system of claim 19, wherein the plurality of multi-function combination ports includes a first combination port configured to fluidly communicate with a drain port and heater port, a second combination port configured to fluidly communicate with a first supply port and second supply port, and a third combination drain port configured to fluidly communicate with a third supply port and a patient port.
